(19) United States
(12) Patent Application Publication
(10) Pub. No.: US 2024/0390868 A1
(43) Pub. Date: Nov. 28, 2024

(54) PRODUCTION SYSTEM AND METHOD FOR PRODUCING NANOPARTICLES

(71) Applicant: SHANGHAI BEST-LINK BIOSCIENCE, LLC, Shanghai (CN)

(72) Inventors: Fuyao ZHANG, Shanghai (CN); Jiaxun WAN, Shanghai (CN)

(21) Appl. No.: 18/694,390

(22) PCT Filed: Sep. 30, 2022
(86) PCT No.: PCT/CN2022/123347

§ 371 Date: Mar. 21, 2024

(30) Foreign Application Priority Data
Sep. 30, 2021 (CN) 202111178935.5

(51) Int. Cl. B01F 23/45; A61K 9/51; B01D 61/14; B01F 25/21; B01F 101/00; B01F 101/56

(52) U.S. Cl. CPC B01F 23/45; A61K 9/51; B01D 61/145; B01F 25/21; B01F 2101/2202; B01F 2101/56

(57) ABSTRACT

Disclosed in the present invention are a production system and method for producing nanoparticles. Particularly disclosed is a production system, which comprising (1) a first pipeline, (2) a second pipeline, (3) a combined pipeline, and a fluid outlet; wherein the first pipeline and the second pipeline are connected to the combined pipeline, a first phase solution enters the combined pipeline through an outlet of the first pipeline, a second phase solution enters the combined pipeline through an outlet of the second pipeline, and the first phase solution and the second phase solution are mixed in the combined pipeline to form a combined phase; which combined phase flows out through the outlet of the combined pipeline. The production system and method of the present invention can realize the continuous, large-scale and controllable production of composite nanoparticles for a photosensitizer/an anti-tumor drug.

### SPECIFICATION

This application claims priority to Chinese Application No. 2021111789355, filed Sep. 30, 2021. The entire disclosures of the above applications are incorporated herein by reference.

### TECHNICAL FIELD

The present invention relates to the field of biomedical technology, specifically relates to a production system and method for producing nanoparticles. The related production method comprises two-phase solutions containing a photosensitizer and an anti-tumor drug combined in the combined pipeline to form a combined phase, and mixed sufficiently through a turbulent mixing device to form a stable dispersed composite nanoparticles of photosensitizer and anti-tumor drug, with a certain particle size and distribution coefficient.

The present invention belongs to the field of nano drug formulation technology.

### BACKGROUND

During the treatment of tumors, there are a variety of therapeutic options to prolong the survival of patients. Anti-tumor drugs used in the treatment include chemotherapy drugs, targeted drugs, photosensitive molecules, photothermal molecules, peptides, proteins, siRNAs, etc. Among them, some drugs need to be made into nano formulations to achieve the anti-tumor effect. Commonly used nano formulations include: nanoparticles, liposomes, polymeric micelles, dendrimers, etc.

Photosensitizer/anti-tumor drug composite nanoparticles can not only play a single therapeutic effect of anti-tumor agent, but also play synergistic roles in the tumor treatment. The following photosensitizer/anti-tumor drug composite nanoparticles have been reported in the literature: Ce6-DOX composite nanoparticles (ACS Appl. Mater. Interfaces 2016, 8, 13262-13269), Ce6-HCPT composite nanoparticles (Nanoscale, 2017, 9, 14347), Ce6-SN-38 composite nanoparticles (Colloids and Surfaces B: Biointerfaces, 2020, 188,110722), ICG-PTX composite nanoparticles (J. Mater. Chem. B, 2019, 7, 6914), ICG-PTX-UA composite nanoparticles (ACS Appl. Mater. Interfaces 2017, 9, 43508), UA-LA-ICG composite nanoparticles (Acta Biomaterialia, 2018, 70, 197), SN-38/ICG composite nanoparticles (CN108159422B). Due to the complexity of the production method of such composite nanoparticles, the reproducibility of the preparation process is poor. Therefore, it is difficult to achieve scale-up and industrial production, which limits the clinical use of these photosensitizer/anti-tumor composite nanoparticles. Therefore, the continuous, large-scale and controllable production method of photosensitizer/anti-tumor composite nanoparticles is urgently needed.

The current large-scale production methods of nano formulations include high-pressure homogenization method, high-shear emulsification method, microfluidization homogenization method, etc. However, these production methods are batch preparation, which have the disadvantages of large amount of process control parameters, poor inter-batch reproducibility and difficulty in scale-up production.

Compared with the batch production methods, the continuous preparation method of nano formulations can produce uniform nanoparticles with adjustable sizes during continuous operation. Besides, the quality of nano formulations could be monitored in real time during the production process, and the nano formulations meeting the quality standards can be collected in real time.

For example, for liposome systems, continuous production can be performed using ethanol injection method. A microfluidic device of Precision NanoSystems in Canada can be used for the continuous production of liposomes (Langmuir, 2012, 28, 3633), the laminar flow of lipid organic phase and aqueous phase were rapidly mixed in a staggered mixer, and liposomes could be produced continuously by parameter optimization. However, since the microfluidic flow channel is usually in the micrometer scale, the fluid flow is in laminar conditions, the single-channel yield is extremely small, and the scale-up production requires multi-channel paralleling. The consistency of each liposome production unit needs to be controlled. The device for continuous preparation of blank liposomes using ethanol injection method reported in the literature (Pharm Res., 2016, 33, 404-416) and patent (CN107427791) can prepare blank liposomes with low polydispersity index by adjusting the flow velocity of ethanol phase and aqueous phase.

For polymer/drug nanoparticle systems, the continuous production can be achieved by flash nanoprecipitation (FNP) (U.S. Pat. No. 10,940,118B2, CN108137819, CN108542894). Nanoprecipitation method is based on the principle of kinetic control and can realize rapid production of nanoparticles by mixing turbulent fluids. This method has the characteristics of high drug loading, short production time (millisecond level), easy control of the size, easy to scale up and continuous production. The principle of preparing polymer/drug nanoparticles by nanoprecipitation method is as follows: the carrier or stabilizer (usually amphiphilic polymers) and hydrophobic drug dissolved in solvent miscible with water to form a homogeneous solution. Then, the solution is rapidly mixed with the anti-solvent (usually water) in a fixed channel. Since the hydrophobic substance is highly supersaturated in the mixed solvent, it is rapidly nucleated in water, and interacts with the polymer (usually an amphiphilic block polymer) at the same time. The polymer encapsulates the nucleating core to form polymer nanoparticles, protecting the nanoparticles from reaggregation, thereby forming nanoparticles with good aqueous dispersion (Expert Opin. Drug Deliv., 2009, 6, 865). The production devices used in the nanoprecipitation method include: confined impinging jet mixer (CIJM) (Physical Review Letters, 2003, 91, 118301; AIChE Journal, 2003, 49, 2264), multi-inlet vortex mixer, (MIVM) (Mol. Pharm., 2013, 10, 4367; Angew. Chem. Int. Ed. Engl., 2021, 60, 15590).

However, compared with liposomes and polymer nanoparticles, the microstructure of small molecule photosensitizer/anti-tumor drug composite nanoparticles is very different: liposomes have a bilayer phospholipid structure; and polymer nanoparticles are core-shell structure of hydrophobic drugs coated with amphiphilic block polymers; while small molecule photosensitizer/anti-tumor drug composite nanoparticles are self-stable photosensitizer/anti-tumor drug nanoaggregates. Therefore, whether nanoprecipitation method is suitable for the preparation of small molecule photosensitizer/anti-tumor drug composite nanoparticles is unpredictable, and many experiments are required for research, verification and optimization. So far, no reports of continuous production of photosensitizer/anti-tumor composite nanoparticles by nanoprecipitation method have been reported.

Based on the preparation of polymer/drug composite nanoparticles by nanoprecipitation method, this application has conducted repeated research and experiments to develop a preparation system for the large-scale production of photosensitizer/anti-tumor drug composite nanoparticles, which is suitable for continuous and controllable scale-up production of photosensitizer/anti-tumor drug composite nanoparticles.

### CONTENT OF THE PRESENT INVENTION

The present invention is intended to overcome the defects and shortcomings of the existing photosensitizer/anti-tumor drug composite nanoparticle production technologies, and provide a production system and production method for producing nanoparticles, which is suitable for continuous scale-up production of photosensitizer/anti-tumor drug composite nanoparticles.

The present invention provides a production system for the continuous, large-scale and controllable production of photosensitizer/anti-tumor drug composite nanoparticles, which is suitable for the production of nano formulations including but not limited to, nano formulations in the particle size range of 1-1000 nm, and the nano formulations are photosensitizer/anti-tumor drug composite nanoparticles.

The present invention provides a (continuous, large-scale and controllable) production system (for producing photosensitizer/anti-tumor drug composite nanoparticles), which includes (1) a first pipeline, (2) a second pipeline, (3) a combined pipeline and its (fluid) outlets;

Wherein, the first pipeline and the second pipeline are connected to the combined pipeline, the first phase solution enters the combined pipeline through the first pipeline outlet, the second phase solution enters the combined pipeline through the second pipeline outlet, and the first phase solution and the second phase solution are mixed in the combined pipeline to form a combined phase; the combined phase flows out through the outlet of the combined pipeline.

In some embodiments, the core part of the production system includes: (1) a first pipeline; (2) a second pipeline; (3) a combined pipeline; (4) turbulent mixing device; (5) fluid outlet;

Wherein, the first pipeline and the second pipeline are connected to the combined pipeline, the first phase solution enters the combined pipeline through the first pipeline outlet, the second phase solution enters the combined pipeline through the second pipeline outlet, the first phase solution and the second phase solution are mixed in the combined pipeline to form a combined phase. The combined phase is fully mixed by a turbulent mixing device. After the mixing, the composite nanoparticles are collected into a suitable container through the outlet of the combined pipeline.

In some embodiments, the first pipeline is coaxial with the combined pipeline and the second pipeline is perpendicular to the combined pipeline.

In some embodiments, the mixing is turbulent mixing. The turbulent mixing can be achieved by adding a turbulent mixing device in the combined pipeline. There can be one or more turbulent mixing devices.

In some embodiments, the first pipeline outlet is positioned within the combined pipeline.

In some embodiments, the first pipeline outlet is a spray hole with a certain shape and diameter, and the first phase solution passes through the first pipeline and enters the combined pipeline through the spray hole.

In some embodiments, the spray hole diameter D₁(S) at the end of first pipeline is selected from 0.03-5.0 mm; the second pipeline inner diameter D₂(IN) is selected from 0.3-50.0 mm; the combined pipeline inner diameter D₃(IN) is selected from 0.3-50.0 mm.

In some embodiments, the combined pipeline length (i.e., combined phase length) is selected from 6 to 120 cm, such as 9 cm.

In some embodiments, the ratio of the combined pipeline length to the combined pipeline inner diameter is (16-17): 1, such as 16.7:1.

In some embodiments, the first pipeline outer diameter D₁(O) is 2 mm.

In some embodiments, the spray hole diameter D₁(S) at the end of first pipeline is 0.3 to 0.6 mm, such as 0.3 mm or 0.6 mm.

In some embodiments, the second pipeline outer diameter D₂(O) is 6 mm.

In some embodiments, the second pipeline inner diameter D₂(IN) is 5.4 mm.

In some embodiments, the combined pipeline outer diameter D₃(O) is 6 mm.

In some embodiments, the combined pipeline inner diameter D₃(IN) is 5.4 mm.

In some embodiments, the second pipeline inner diameter D₂(IN) is the same as the combined pipeline inner diameter D₃(IN).

In some embodiments, the ratio of the spray hole diameter D₁(S) at the end of first pipeline to the combined pipeline inner diameter D₃(IN) can be 1:(2-50), such as 1:9 or 1:18.

In some embodiments, the turbulent mixing device is a device that mixes the first phase solution and the second phase solution to reach a turbulent flow state, such as a static mixer. The static mixer can be selected from one or more of SV type static mixer, SX type static mixer, SL type static mixer, SH type static mixer and SK type static mixer, preferably SK type static mixer.

In some embodiments, the materials used in the first pipeline, the second pipeline, the combined pipeline, the turbulent mixing device, and the fluid outlet are each selected from one or more of stainless steel, polytetrafluoroethylene, polyethylene, polypropylene, latex, silicone, or other polymer materials.

In some embodiments, the turbulent mixing can cause the fluid in the combined phase to reach a turbulent transition state or turbulent flow state by increasing the fluid flow velocity. The Reynolds number in the combined phase depends on the smoothness of the circular stainless steel pipe wall. For example, when the pipe wall is rough, a lower Reynolds number can also achieve turbulent mixing conditions (such as Re between 500-2000, this range is usually considered to be a laminar flow conditions).

In some embodiments, the turbulent mixing can be achieved by making the combined phase a bent pipe with a certain curvature, by changing the direction of fluid flow, enhancing the convection of the fluid, and enhancing the mixing of the fluid. At this time, in addition to being greater than 4000, the Reynolds number in the combined phase calculated based on the fluid in the circular tube can also be between 500-4000.

In some embodiments, the turbulent mixing can be achieved by equipping a static mixer in the combined phase. The static mixer can include but is not limited to: SV type static mixer, SX type static mixer, SL type static mixer, SH type Static mixers, SK type static mixers, etc. that can divide the fluid through turbulent mixing elements, change the flow direction of the fluid, enhance the convection of the fluid, and increase the mixing of the fluid. Under these circumstances, in addition to being greater than 4000, the Reynolds number in the combined phase calculated based on the fluid in the circular tube can also be between 500-4000.

In some embodiments, the quantity of flow Q₁ of the first phase solution through the first pipeline is selected from 1-1000 ml/min; wherein the temperature T₁ of the first phase solution is selected from 0-90° C.; the quantity of flow Q₂ of the second phase solution through the second pipeline is selected from 10-10000 ml/min; the temperature T₂ of the second phase solution is selected from 0-90° C.

The present invention also provides a (continuous, large-scale and controllable) production method for producing photosensitizer/anti-tumor drug composite nanoparticles, which includes the following steps: In a production system as described above, a first phase solution and a second phase solution are mixed, and the resulting photosensitizer/anti-tumor drug composite nanoparticles are collected from combined phase through the fluid outlet;

The solvent in the first phase solution is a good solvent for an anti-tumor drug or its pharmaceutically acceptable salt, and the solute is (1) an anti-tumor drug or its pharmaceutically acceptable salt, and a photosensitizer, or (2) an anti-tumor drug or its pharmaceutically acceptable salt;

The solvent in the second phase solution is an anti-solvent of the anti-tumor drug or its pharmaceutically acceptable salt, and the solute is (1) absent, or (2) a photosensitizer;

When the solute in the first phase solution is an anti-tumor drug or its pharmaceutically acceptable salt, and a photosensitizer, the solute in the second phase solution does not exist;

When the solute in the first phase solution is an anti-tumor drug or its pharmaceutically acceptable salt, the solute in the second phase solution is a photosensitizer.

In some embodiments, the production methods include:
a) One or more of the anti-tumor drugs or their pharmaceutically acceptable salts and one or more of the photosensitizers are dissolved in a first phase solvent to form a first phase solution, and the first phase solution is a good solvent for the anti-tumor drug or its pharmaceutically acceptable salt, and the photosensitizer;
b) The second phase solution is an anti-solvent for anti-tumor drugs or their pharmaceutically acceptable salts and photosensitizers;
c) The first phase solution with the quantity of flow Q₁ and the second phase solution with the quantity of flow Q₂ are mixed in the combined phase. Under the action of turbulent shear, the two-phase solutions mix rapidly to form a mixed solvent of the first phase and the second phase and produce a stably dispersed composite nano formulation of photosensitizer and anti-tumor drug with a certain particle size and distribution coefficient.

Wherein, during the mixing process of the first phase solution and the second phase solution in the combined phase, under the action of turbulent shear, the two-phase solutions are rapidly mixed. The anti-tumor drug or its pharmaceutically acceptable salt dissolved in the first phase solution reaches a highly supersaturated state in the combined phase, and undergoes rapid nucleation in the mixed solvent. The core of anti-tumor drug or its pharmaceutically acceptable salt grows while interacting with the photosensitizer to form a composite nano formulation of photosensitizer and anti-tumor drug that is stably dispersed in the mixed solvent of the first phase and the second phase with a certain particle size and distribution coefficient. The obtained photosensitizer and anti-tumor drug composite nano formulation is further purified by ultrafiltration to remove pharmaceutically unusable solvents, added a freeze-drying protective agent, filtered, sterilize, filled and freeze-dried under aseptic conditions to obtain different clinically available nano formulations.

In some embodiments, the production method includes:
a) One or more of the anti-tumor drugs or their pharmaceutically acceptable salts are dissolved in first phase solvent to form first phase solution, and the first phase solvent is good solvent for anti-tumor drugs or their pharmaceutically acceptable salts;
b) One or more of the photosensitizers are dissolved in second phase solvent to form second phase solution, and the second phase solvent is anti-solvent for anti-tumor drugs or their pharmaceutically acceptable salts;
c) The first phase solution with quantity of flow Q₁ and the second phase solution with quantity of flow Q₂ are mixed in the combined phase. Under the action of turbulent shear, the two-phase solutions mix rapidly to form a mixed solvent of the first phase and the second phase and produce a stably dispersed composite nano formulation of photosensitizer and anti-tumor drug with a certain particle size and distribution coefficient.

In some embodiments, the temperature of the first phase solution is 0-90° C., such as 25° C.

In some embodiments, the temperature of the second phase solution is 0-90° C., such as 25° C.

In some embodiments, the fluid Reynolds number Re of the combined phase is 800 to 7700, such as 839, 1934, 2579, 3868, 5158, 5236, 5539, 5841, 5236, 6477, 6750, 7053, 7356 or 7659), preferably 3000 to 7700, more preferably 3868 to 7659.

In some embodiments, the flow velocity ratio FVR between the first phase solution and the combined phase is 5 to 26 (e.g., 5.2, 5.8, 17.3, 18.1, 18.9, 19.8, 20.8, 21.9, 23.1, 24.4 or 26), preferably 17 to 26, more preferably 20.8 to 26.

In some embodiments, when the Re of the combined phase is less than 3000, the FVR of the production system is 17 to 26, and/or the production system further includes a static mixer.

In some embodiments, when the Re of the combined phase is less than 3868, the FVR of the production system is 20.8 to 26, and/or the production system further includes a static mixer.

In some embodiments, when the FVR of the production system is less than 17, the Re of the combined phase is 3000 to 7700, and/or the production system further includes a static mixer.

In some embodiments, when the FVR of the production system is less than 20.8, the Re of the combined phase is 3868 to 7659, and/or the production system further includes a static mixer.

In some embodiments, the photosensitizer includes one or more of IR780, IR820, indocyanine green or indocyanine green analogs; the porphyrin type molecules are selected from hematoporphyrin monomethyl ether; the porphyrin precursor is selected from one of 5-aminolevulinic acid and/or 5-aminolevulinic acid esters; the phthalocyanine type molecules are selected from copper phthalocyanine, cobalt phthalocyanine, aluminum phthalocyanine, nickel phthalocyanine, calcium phthalocyanine, sodium phthalocyanine, magnesium phthalocyanine, zinc phthalocyanine, indium phthalocyanine, oxytitanium phthalocyanine, manganese phthalocyanine, or one or more of the phthalocyanine derivatives; the chlorin type molecules are selected from one or more of chlorin, talaporfin, verteporfin, temoporfin, rostaporfin, porfimer sodium, hemoporfin and HPPH.

In some embodiments, the photosensitizer is selected from one or more of cyanine type molecules, porphyrin type molecules, porphyrin precursors, phthalocyanine type molecules and chlorin type molecules; wherein, the cyanine type molecules are preferably selected from one or more of indocyanine green (IR780), new indocyanine green (IR820), indocyanine green and indocyanine green analogs; the porphyrin type molecule is preferably selected from hematoporphyrin monomethyl ether; the porphyrin precursor is preferably selected from 5-aminolevulinic acid and/or 5-aminolevulinic acid esters; the phthalocyanine type molecule is preferably selected from one or more of copper phthalocyanine, cobalt phthalocyanine, aluminum phthalocyanine, nickel phthalocyanine, calcium phthalocyanine, sodium phthalocyanine, magnesium phthalocyanine, zinc phthalocyanine, indium phthalocyanine, oxytitanium phthalocyanine, manganese phthalocyanine or phthalocyanine derivatives; the chlorin type molecules are preferably selected from one or more of chlorins, talaporfin, verteporfin, temoporfin, rostaporfin, porfimer sodium, hemoporfin and HPPH.

In some embodiments, the photosensitizer is indocyanine green or chlorin e6.

In some embodiments, the anti-tumor drug contains one or more of aromatic rings or aromatic heterocycles in its structure. The anti-tumor drugs are selected from one or more of camptothecin type compounds, paclitaxel type compounds, anthracycline type compounds, targeted drugs or other anti-tumor drugs; wherein the camptothecin type molecules are preferably selected from one or more of camptothecin, 9-aminocamptothecin, 9-nitrocamptothecin, lurtotecan, gimatecan, belotecan, 10-hydroxycamptothecin, 10-hydroxy-7-ethyl-camptothecin (SN-38), exatecan, topotecan and deruxtecan; the paclitaxel type compounds are preferably selected from one or more of paclitaxel, docetaxel, cabazitaxel, 7-epipaclitaxel, 2′-acetylpaclitaxel, 10-deacetylpaclitaxel, 7-epi-10-deacetyltaxol, 7-xylosyltaxol, 10-deacetyl-7-glutarylpaclitaxel, 7-N,N-dimethylglycylpaclitaxel, 7-L-alanylacetaxel and larotaxel; the anthracycline type compounds are preferably selected from one or more of doxorubicin, epirubicin, daunorubicin, pirarubicin and aclacinomycin; the targeted drugs are preferably selected from one or more of gefitinib, erlotinib, lapatinib, afatinib, dacomitinib, vandetanib, neratinib, osimertinib, imatinib, sorafenib, sunitinib, lapatinib, dasatinib, olaparib, niraparib, rucaparib, fluzoparib, pamiparib, veliparib, talazoparib and apatinib; the other anti-tumor drugs are preferably selected from one or more of etoposide, teniposide, vinblastine, vincristine, vinorelbine, vindesine, maytansine, curcumin, harringtonine, homoharringtonine, gemcitabine, capecitabine, fludarabine, cladribine, pemetrexed, bortezomib, carfilzomib, ixazomib, carmustine, fluorouracil, cytarabine, cyclosporine A, eribulin and trabectedin.

In some embodiments, the anti-tumor drug is camptothecin, 10-hydroxycamptothecin, exatecan, Dxd, paclitaxel, sorafenib or curcumin.

In some embodiments, in the photosensitizer/anti-tumor drug composite nanoparticles, the combination of the photosensitizer and the anti-tumor drug is a combination of indocyanine green and camptothecin, indocyanine green and 10-hydroxycamptothecin, indocyanine green and 7-ethylcamptothecin, indocyanine green and 7-ethyl-10-hydroxycamptothecin, indocyanine green and exatecan, indocyanine green and Dxd, indocyanine green and paclitaxel, indocyanine green and docetaxel, indocyanine green and cabazitaxel, indocyanine green and sorafenib, indocyanine green and curcumin or chlorin e6 and 7-ethyl-10-hydroxycamptothecin;

The preferable combination of the photosensitizer and the anti-tumor drug is a combination of indocyanine green and camptothecin, indocyanine green and 10-hydroxycamptothecin, indocyanine green and exatecan, indocyanine green and Dxd, indocyanine green and paclitaxel, indocyanine green and sorafenib, indocyanine green and curcumin, indocyanine green and 7-ethyl-10-hydroxycamptothecin or chlorin e6 and 7-ethyl-10-hydroxycamptothecin;

In some embodiments, the range of anti-tumor drug/(anti-tumor drug+photosensitizer) in the composite nanoparticles is 0.1-0.9, such as 10%, 15%, 20%, 25%, 30%, 35%, 40%, 45%, 50%, 55%, 60%, 65%, 70%, 75%, 80%, 85%, 90%.

In some embodiments, the molar ratio of the anti-tumor drug or its pharmaceutically acceptable salt to the photosensitizer is (1-15):1, such as 1:1, 2:1, 5:1, 6:1, 7:1, 8:1, 10:1 or 15:1.

In some embodiments, when the anti-tumor drug is 7-ethyl-10-hydroxycamptothecin and the photosensitizer is indocyanine green, the molar ratio of the anti-tumor drug or its pharmaceutically acceptable salt to the photosensitizer is (2-15):1, such as 2:1, 5:1, 10:1 or 15:1.

In some embodiments, when the anti-tumor drug is camptothecin and the photosensitizer is indocyanine green, the molar ratio of the anti-tumor drug or its pharmaceutically acceptable salt to the photosensitizer is (1-10):1, such as 1:1, 2:1, 5:1 or 10:1.

In some embodiments, when the anti-tumor drug is 10-hydroxycamptothecin and the photosensitizer is indocyanine green, the molar ratio of the anti-tumor drug or its pharmaceutically acceptable salt to the photosensitizer is (1-10):1, such as 1:1, 2:1, 5:1 or 10:1.

In some embodiments, when the anti-tumor drug is exatecan and the photosensitizer is indocyanine green, the molar ratio of the anti-tumor drug or its pharmaceutically acceptable salt to the photosensitizer is (2-15):1, preferably (2-10):1, such as 2:1, 5:1 or 10:1.

In some embodiments, when the anti-tumor drug is Dxd and the photosensitizer is indocyanine green, the molar ratio of the anti-tumor drug or its pharmaceutically acceptable salt to the photosensitizer is (5-10):1, such as 5:1 or 10:1.

In some embodiments, when the anti-tumor drug is sorafenib and the photosensitizer is indocyanine green, the molar ratio of the anti-tumor drug or its pharmaceutically acceptable salt to the photosensitizer is (6-8):1, such as 6:1 or 8:1.

In some embodiments, when the anti-tumor drug is paclitaxel and the photosensitizer is indocyanine green, the molar ratio of the anti-tumor drug or its pharmaceutically acceptable salt to the photosensitizer is (1-10):1, such as 1:1, 2:1, 5:1 or 10:1.

In some embodiments, when the anti-tumor drug is curcumin and the photosensitizer is indocyanine green, the molar ratio of the anti-tumor drug or its pharmaceutically acceptable salt to the photosensitizer is (5-8):1, such as 5:1, 6:1, 7:1 or 8:1.

In some embodiments, when the anti-tumor drug is 7-ethyl-10-hydroxycamptothecin and the photosensitizer is chlorin e6, the molar ratio of the anti-tumor drug or its pharmaceutically acceptable salt to the photosensitizer is 2:1.

In some embodiments, the solvent used in the first phase solution and the second phase solution is water, an aqueous buffer solution with a certain pH value, or an organic solvent miscible with water. Further, the organic solvent is one or more of methanol, ethanol, propanol, isopropanol, butanol, isobutanol, tert-butanol, DMF, DMAc, N-methylpyrrolidone, DMSO, butyl sulfone, tetramethylene sulfone, THF, 2-methyltetrahydrofuran, acetonitrile, acetone, ethylene glycol, ethylene glycol methyl ether, ethylene glycol ethyl ether, HMPA, dioxane, formic acid, acetic acid, hydroxypropionic acid, ethylamine, ethylenediamine, glycerol or pyridine.

In some embodiments, the solvent in the first phase solution is selected from one or more of methanol, ethanol, propanol, isopropanol, butanol, isobutanol, tert-butanol, DMF, DMAc, N-methylpyrrolidone, DMSO, butyl sulfone, tetramethylene sulfone, THF, 2-methyltetrahydrofuran, acetonitrile, acetone, ethylene glycol, ethylene glycol methyl ether, ethylene glycol ethyl ether, HMPA, dioxane, formic acid, acetic acid, hydroxypropionic acid, ethylamine, ethylenediamine, glycerol and pyridine, preferably DMSO.

In some embodiments, the solvent in the second phase solution is water or a buffer with a pH of 2-10, preferably water.

In some embodiments, the molar concentration of the anti-tumor drug or its pharmaceutically acceptable salt in the first phase solution is 0.01-0.3M; preferably 0.05-0.1M, such as 0.05M or 0.1M.

In some embodiments, the molar concentration of the photosensitizer in the first phase solution or the second phase solution is 0.01-0.3M; preferably 0.05-0.1M, such as 0.05M or 0.1M.

In some embodiments, the concentration of the anti-tumor drug in the first phase solution ranges from 0.1 to 200 mg/ml, for example, it can be 0.1 mg/ml, 1 mg/ml, 5 mg/ml, 10 mg/ml, 20 mg/ml, 40 mg/ml, 60 mg/ml, 80 mg/ml, 100 mg/ml, 120 mg/ml, 140 mg/ml, 160 mg/ml, 180 mg/ml, 200 mg/ml, preferably 10-100 mg/ml.

In some embodiments, the concentration of the photosensitizer in the first phase solution or the second phase solution ranges from 0.1 to 200 mg/ml, for example, it can be 0.1 mg/ml, 1 mg/ml, 5 mg/ml, 10 mg/ml, 20 mg/ml, 40 mg/ml, 60 mg/ml, 80 mg/ml, 100 mg/ml, 120 mg/ml, 140 mg/ml, 160 mg/ml, 180 mg/ml, 200 mg/ml, preferably 10-100 mg/ml.

In some embodiments, the production method also includes the following post-reaction processing steps: adding water to the prepared mixed solution and ultrafiltration; or further concentration and ultrafiltration.

Wherein, the ultrafiltration can use a 2-100 kDa ultrafiltration membrane, preferably a 30 kDa ultrafiltration membrane.

In some embodiments, nanoparticles are prepared with an (average) particle size of less than 1000 nm.

In some embodiments, nanoparticles are prepared with an (average) particle size of less than 500 nm.

In some embodiments, nanoparticles are prepared with an (average) particle size of less than 200 nm.

In some embodiments, nanoparticles are prepared with an (average) particle size of 50-200 nm.

In some embodiments, nanoparticles are prepared with a polydispersity index of less than 0.3.

In some embodiments, nanoparticles are prepared with a polydispersity index of less than 0.2.

In some embodiments, nanoparticles are prepared with a polydispersity index of less than 0.1.

In some embodiments, the SN-38 encapsulation efficiency of the prepared SN-38/ICG nanoparticles in the 15-20 ml scale is 98.2%, and the nanoparticle diameter is 114±6 nm; the SN-38 encapsulation efficiency in the 60 ml scale is 91.7%, and the particle size of the nanoparticles is 127±6 nm; the encapsulation efficiency of SN-38 in the 557 ml scale is 89.2%, and the particle size of the nanoparticles is 129±10 nm; the encapsulation efficiency of SN-38 can be maintained to be greater than 80% during the scale-up process.

The present invention also provides a photosensitizer/anti-tumor drug composite nanoparticle, which is prepared by the above production method.

Compared with the prior art, the present invention has the following advantages:
i. The present invention solves the current problem that photosensitizer/anti-tumor drug composite nanoparticles cannot be produced continuously on a large scale, and provides a method that can realize continuous, large-scale and controllable production of photosensitizer/anti-tumor drug composite nanoparticles. The production system can be used for the continuous production of nano formulations with particle sizes ranging from 1 to 1000 nm.
ii. The production system and method for the continuous, large-scale and controllable production of photosensitizer/anti-tumor drug composite nanoparticles provided by the present invention are general and suitable for the continuous production of composite nanoparticles with different combinations of photosensitizers and anti-tumor drugs.
iii. The present invention provides a method for preparing photosensitizer/anti-tumor drug composite nanoparticles, which has good reproducibility, can be scaled up step by step and can achieve the production with simple equipment and operation. This method can produce nanoparticles with controllable size and low polydispersity index on a large scale.

### BRIEF DESCRIPTION OF THE DRAWINGS

FIG. 1. Schematic diagram of the core part of the production system for the continuous production of photosensitizers and anti-tumor drug composite nano formulations (D₁(S)=0.3 mm, D₃(IN)=5.4 mm)

FIG. 2. Schematic diagram of the core part of the production system for the continuous production of photosensitizers and anti-tumor drug composite nano formulations (D₁(S)=0.3 mm, D₃(IN)=5.4 mm, SK static mixer)

FIG. 3. Particle size and distribution of SN-38/ICG nanoparticles (Example 5)

FIG. 4. Particle size and distribution of SN-38/ICG nanoparticles (Example 6)

FIG. 5. Particle size and distribution of SN-38/ICG nanoparticles (Example 7)

FIG. 6. Particle size and distribution of SN-38/ICG nanoparticles (Example 8)

FIG. 7. Particle size and distribution of SN-38/ICG nanoparticles (Example 9)

FIG. 8. Particle size and distribution of SN-38/ICG nanoparticles (Example 10)

FIG. 9. Particle size and distribution of SN-38/ICG nanoparticles (Example 11)

FIG. 10. Particle size and distribution of SN-38/ICG nanoparticles (Example 12)

FIG. 11. Particle size and distribution of SN-38/ICG nanoparticles (Example 13)

FIG. 12. Particle size and distribution of SN-38/ICG nanoparticles (Example 14)

FIG. 13. Particle size and distribution of SN-38/ICG nanoparticles (Example 15)

FIG. 14. Particle size and distribution of SN-38/ICG nanoparticles (Example 16)

FIG. 15. Particle size and distribution of SN-38/ICG nanoparticles (Example 17)

FIG. 16. Particle size and distribution of SN-38/ICG nanoparticles (Example 18)

FIG. 17. Particle size and distribution of SN-38/ICG nanoparticles (Example 19)

FIG. 18. Particle size and distribution of SN-38/ICG nanoparticles (Example 20)

FIG. 19. Particle size and distribution of SN-38/ICG nanoparticles (Example 21)

FIG. 20. Particle size and distribution of SN-38/ICG nanoparticles (Example 22)

FIG. 21. Particle size and distribution of SN-38/ICG nanoparticles (Example 23)

FIG. 22. Particle size and distribution of SN-38/ICG nanoparticles (Example 24)

FIG. 23. Particle size and distribution of SN-38/ICG nanoparticles (Example 25)

FIG. 24. Particle size and distribution of SN-38/ICG nanoparticles (Example 26)

FIG. 25. Particle size and distribution of SN-38/ICG nanoparticles (Example 27)

FIG. 26. Particle size and distribution of SN-38/ICG nanoparticles (Example 28)

FIG. 27. Particle size and distribution of SN-38/ICG nanoparticles (Example 29)

FIG. 28. Particle size and distribution of SN-38/ICG nanoparticles (Example 30)

FIG. 29. Particle size and distribution of SN-38/ICG nanoparticles (Example 31)

FIG. 30. Particle size and distribution of SN-38/ICG nanoparticles (Example 32)

FIG. 31. Particle size and distribution of SN-38/ICG nanoparticles (Example 33)

FIG. 32. Particle size and distribution of SN-38/ICG nanoparticles (Example 34)

FIG. 33. Particle size and distribution of SN-38/ICG nanoparticles (Example 35)

FIG. 34. Particle size and distribution of SN-38/ICG nanoparticles (Example 36)

FIG. 35. Particle size and distribution of SN-38/ICG nanoparticles (Example 37)

FIG. 36. Particle size and distribution of SN-38/ICG nanoparticles (Example 38)

FIG. 37. Particle size and distribution of SN-38/ICG nanoparticles (Example 39) before ultrafiltration

FIG. 38. Particle size and distribution of SN-38/ICG nanoparticles (Example 39) after ultrafiltration

FIG. 39. Comparative Example 2 production of SN-38/ICG nanoparticles (SN-38:ICG=10:1-1:1, molar ratio), small amount of SN-38/ICG nanoparticles was prepared by nanoprecipitation method

FIG. 40. Production of SN-38/ICG nanoparticles in Example 40 (SN-38:ICG=15:1-5:1, molar ratio) particle size distribution graph

FIG. 41. Production of camptothecin/ICG nanoparticles in Example 41 (camptothecin:ICG=10:1-1:1, molar ratio) particle size distribution graph

FIG. 42. Production of 10-hydroxycamptothecin/ICG nanoparticles in Example 42 (10-hydroxycamptothecin:ICG=10:1-1:1, molar ratio) particle size distribution graph

FIG. 43. Production of exatecan/ICG nanoparticles in Example 43 (exatecan:ICG=2:1-10:1, molar ratio) particle size distribution graph

FIG. 44. Production of sorafenib/ICG nanoparticles in Example 45 (sorafenib:ICG=1:1-8:1, molar ratio) solution after centrifugation and precipitation

FIG. 45. Production of sorafenib/ICG nanoparticles in Example 45 (sorafenib:ICG=6:1-8:1, molar ratio) particle size distribution graph

FIG. 46. Production of PTX/ICG nanoparticles in Example 46 (PTX:ICG=5:1-10:1, molar ratio) particle size distribution graph

FIG. 47. Production of curcumin/ICG nanoparticles in Example 47 (curcumin:ICG=5:1-8:1, molar ratio) particle size distribution graph

FIG. 48. Production of SN-38/Ce6 nanoparticles in Example 48 (SN-38/Ce6=2:1, molar ratio) chlorin e6 particle size distribution graph

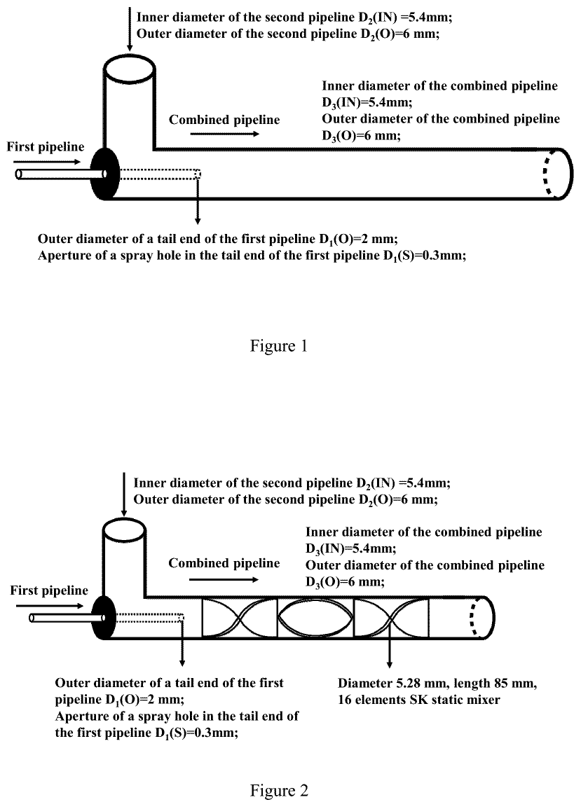

### DETAILED DESCRIPTION OF THE PREFERRED EMBODIMENT

The abbreviations used in the present invention are shown in Table 1.

TABLE 1

| Abbreviation | Full name |
|---|---|
| D₁(S) | Diameter of a spray hole in the end of the first pipeline |
| D₂(IN) | Inner diameter of the second pipeline |
| D₃(IN) | Inner diameter of the combined pipeline |
| Re | Reynolds number |
| Q₁ | The quantity of flow of the first pipeline |
| Q₂ | The quantity of flow of the second pipeline |
| T₁ | First phase solution temperature |
| T₂ | Second phase solution temperature |
| IR780 | 2-[2-[2-Chloro-3-[(1,3-dihydro-3,3-dimethyl-1-propyl-2H- |
| | indol-2-ylidene)ethylidene]-1-cyclohexen-1-yl]ethenyl]-3,3- |
| | dimethyl-1-propylindolium iodide |
| IR820 | New indocyanine green |
| ICG | Indocyanine green |
| HPPH | Photochlor |
| SN-38 | 7-Ethyl-10-hydroxycamptothecin |
| DMF | N,N-dimethylformamide |
| DMAc | N,N-dimethylacetamide |
| DMSO | Dimethyl sulfoxide |
| THF | Tetrahydrofuran |
| HMPA | Hexamethylphosphoramide |
| siRNA | Small interfering RNA |
| Ce6 | Chlorin e6 |
| DOX | Doxorubicin |
| HCPT | 10-Hydroxycamptothecin |
| PTX | Paclitaxel |
| UA | Ursolic acid |
| LA | Lactobionic acid |
| FNP | Flash nanoprecipitation |
| CIJM | Confined impinging jet mixer |
| MIVM | Multi-inlet vortex mixer |
| TEM | Transmission electron microscope |
| FVR | The flow velocity ratio of the first phase solution to the |
| | combined phase solution |

The corresponding structures of materials used in the present invention are shown in Table 2.

TABLE 2

| | |
|---|---|
| SN-38 | 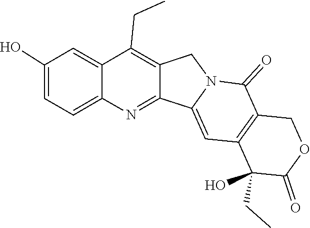 [SMILES: CCc1c2c(nc3ccc(O)cc13)-c1cc3c(c(=O)n1C2)COC(=O)[C@]3(O)CC] |
| ICG | 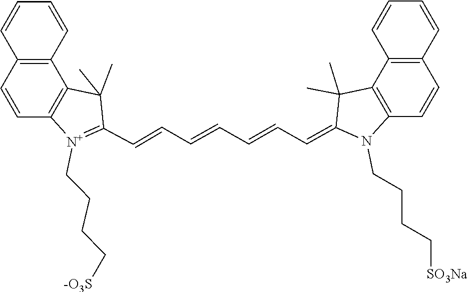 |
| Camptothecin | 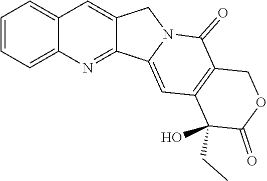 [SMILES: CC[C@@]1(O)C(=O)OCc2c1cc1n(c2=O)Cc2cc3ccccc3nc2-1] |
| 10-Hydroxycamptothecin | 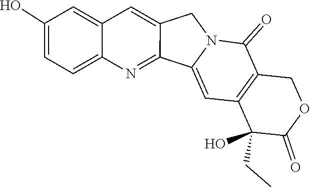 [SMILES: CC[C@@]1(O)C(=O)OCc2c1cc1n(c2=O)Cc2cc3cc(O)ccc3nc2-1] |
| Exatecan | 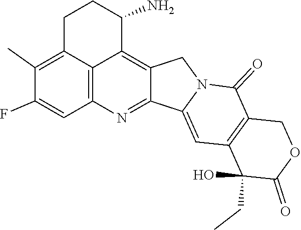 [SMILES: CC[C@@]1(O)C(=O)OCc2c1cc1n(c2=O)Cc2c-1nc1cc(F)c(C)c3c1c2[C@@H](N)CC3] |
| Dxd | 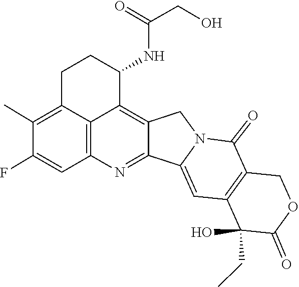 [SMILES: CC[C@@]1(O)C(=O)OCc2c1cc1n(c2=O)Cc2c-1nc1cc(F)c(C)c3c1c2[C@@H](NC(=O)CO)CC3] |

The present invention is further described below by examples, but the present invention is not limited to the scope of the described examples.

Experimental methods that do not indicate specific conditions in the following examples should be selected according to conventional methods and conditions, or according to product specifications.

The endpoints of ranges and any values disclosed herein are not limited to the precise range or value, but these ranges or values are to be understood to include values close to such ranges or values. For numerical ranges, the endpoint values of each range, the endpoint values of each range and individual point values, and the individual point values can be combined with each other to obtain one or more new numerical ranges. These numerical ranges shall be deemed to be specifically disclosed herein.

The turbulent mixing part in this application is a circular pipe with a certain diameter and length, and turbulent flow conditions are achieved through one or more of the following methods:

Increase flow velocity:
Thorough mixing requires turbulence, Re>4000
Quantity of flow Q=πd²/4*υ
Re=ρυd/μ=4ρQ/(πdμ)>4000
When d=0.25 mm
Q>πμd=2.8 L/h
When d=4 mm
Q>45 L/h
When d=40 mm
Q>450 L/h

Wherein, Re is the Reynolds number, Q is the quantity of flow, d is the pipeline diameter, υ is the fluid flow velocity in the pipe, μ is the fluid viscosity, 20° C. μwater=10⁻³ Pa·s

It should be pointed out that for the preparation of certain composite nanoparticles of photosensitizers and anti-tumor drugs, nano-formulations with a certain particle size and particle size distribution can also be obtained with Re in the range of 500-4000.

Change the shape of the pipeline:

By increasing the tortuosity of the pipeline, the direction of fluid flow is forcibly changed, which enhances the fluid mixing.

Add a static mixer to the pipeline:

The static mixer can include but is not limited to: SV type static mixer, SX type static mixer, SL type static mixer, SH type static mixers, SK type static mixers, etc. that can divide the fluid through turbulent mixing elements, change the flow direction of the fluid, enhance the convection of the fluid, and increase the mixing of the fluid.

The SV type static mixer unit is a cylinder assembled from certain regular wave plates.

The SX type static mixer unit consists of many X-shaped units composed of crossed horizontal bars according to certain rules.

The SL type static mixer unit is composed of crossed horizontal bars according to a certain pattern to form a single X-shaped unit.

The SK type static mixer unit is composed of single-channel left and right twisted spiral plates welded together.

SH type static mixer unit is composed of double channels, with a fluid redistribution chamber between the units.

### Example 1: Preparation of First Phase Solution and Second Phase Solution

### Example: Preparation of the First Phase Solution of SN-38:ICG=2:1 (Molar Ratio)

Preparation of the first phase solution: ICG 1.30 g and SN-38 1.30 g were dissolved in 35.1 g DMSO, total weight 37.7 g, SN-38 content: 3.4 wt. %, ICG content: 3.4 wt. %, SN-38 and ICG molar ratio is 2:1.

Unless otherwise specified, the second phase solution is water.

### Example 2: Determination of SN-38 and ICG Concentration

Instrument: Agilent1260 HPLC

Column: Waters XBridge C18 4.6*150 mm, 3.5 μm

Mobile phase: Use 10 mmol/L sodium dihydrogen phosphate solution (adjust to pH 4.0 with phosphoric acid) as phase A, use acetonitrile as phase B, and perform gradient elution according to the following table:

| Time (min) | A % | B % |
|---|---|---|
| 0 | 80 | 20 |
| 10 | 20 | 80 |
| 11 | 80 | 20 |
| 16 | 80 | 20 |

Chromatographic parameters: flow rate: 1 ml/min; column temperature: 35° C.; detection wavelength: 264 nm; injection volume: 10 μl

Diluent: DMSO

Test solution: Use a pipette to accurately take 200 μl of the SN-38/ICG nano-formulation solution, place it in a 10 ml volumetric flask, add DMSO to dissolve and quantitatively dilute to the mark and shake well.

Reference substance solution: Take about 10 mg of ICG or SN-38 reference substance, weigh it accurately, put it in a 100 ml volumetric flask, add DMSO to dissolve it and quantitatively dilute it to the mark and shake well.

Calculation method: Peak area external standard method

Unless otherwise specified, all other compounds are measured using this mobile phase, and the wavelength depends on the specific compound.

### Example 3: Determination of Nanoparticle Size

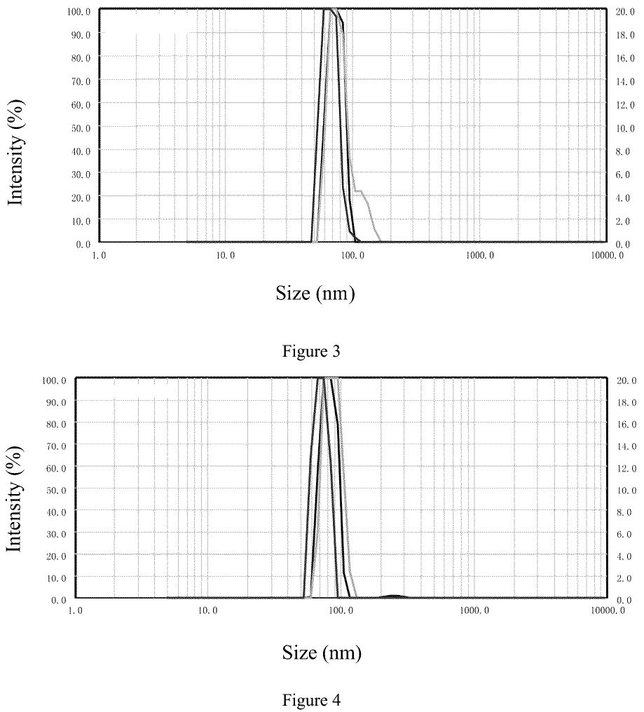

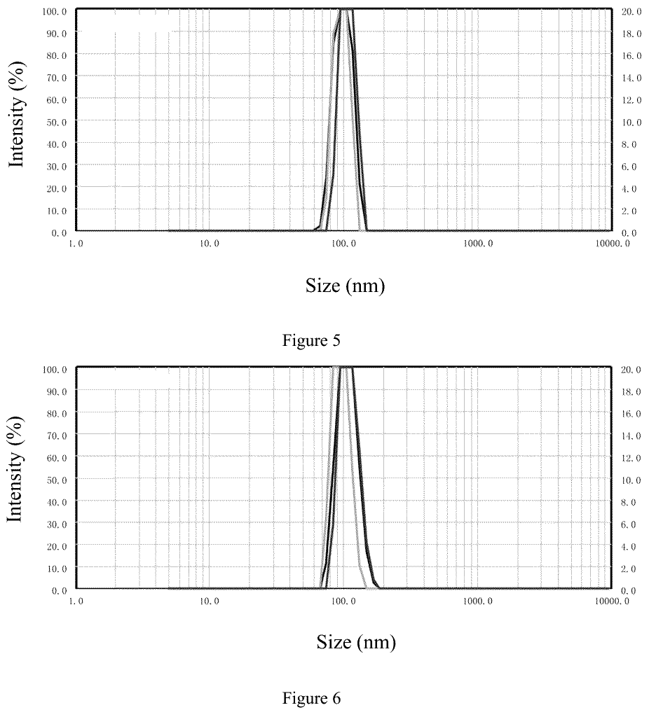

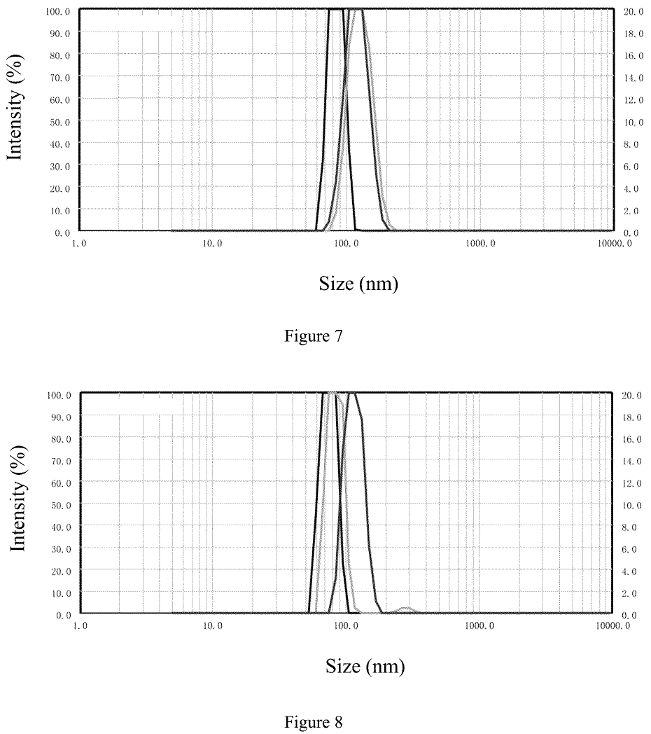

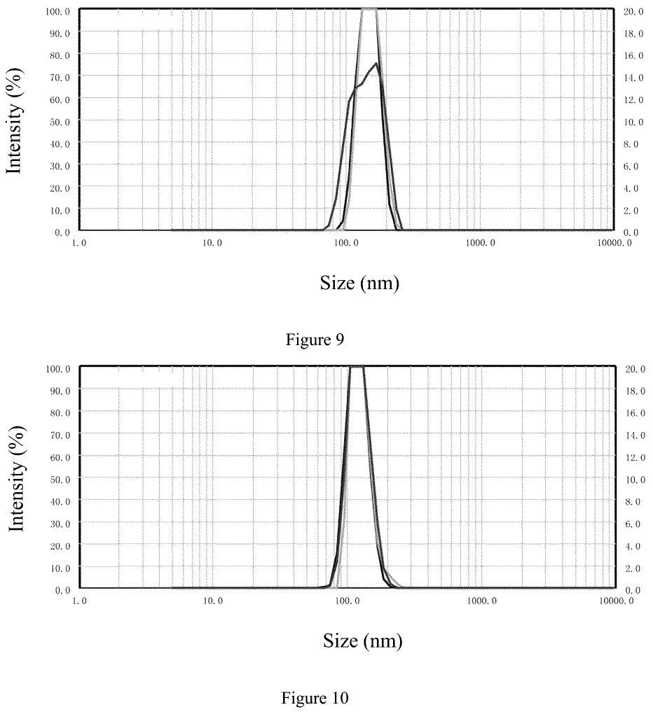

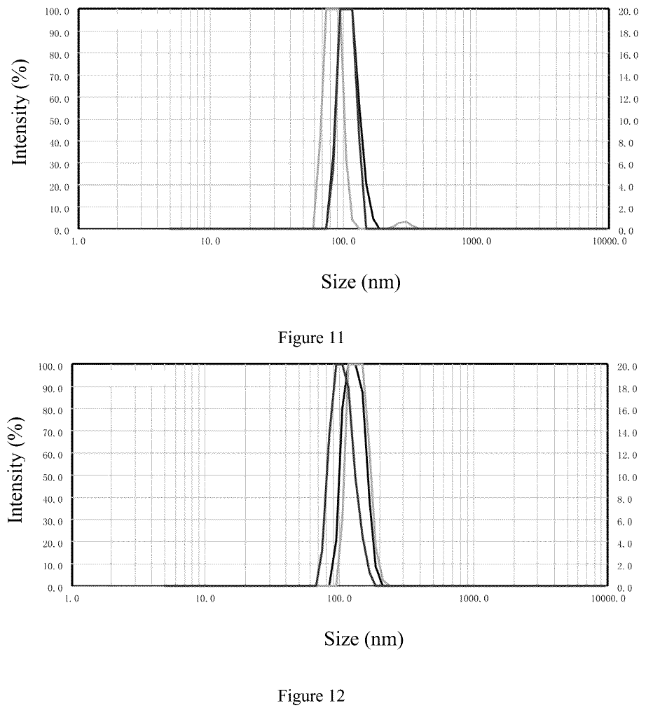

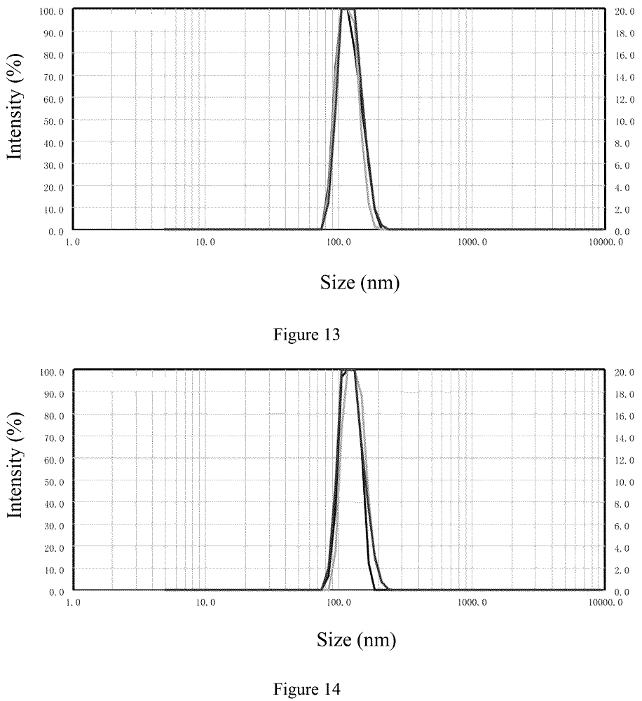

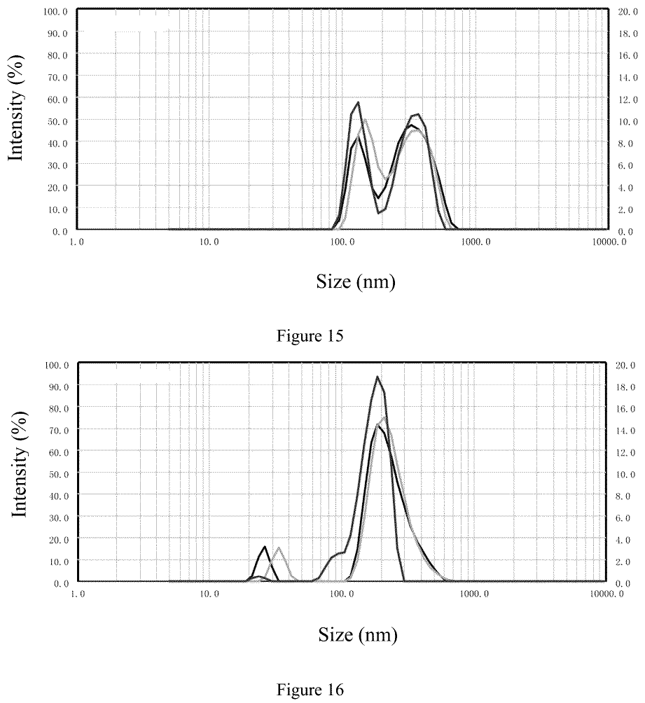

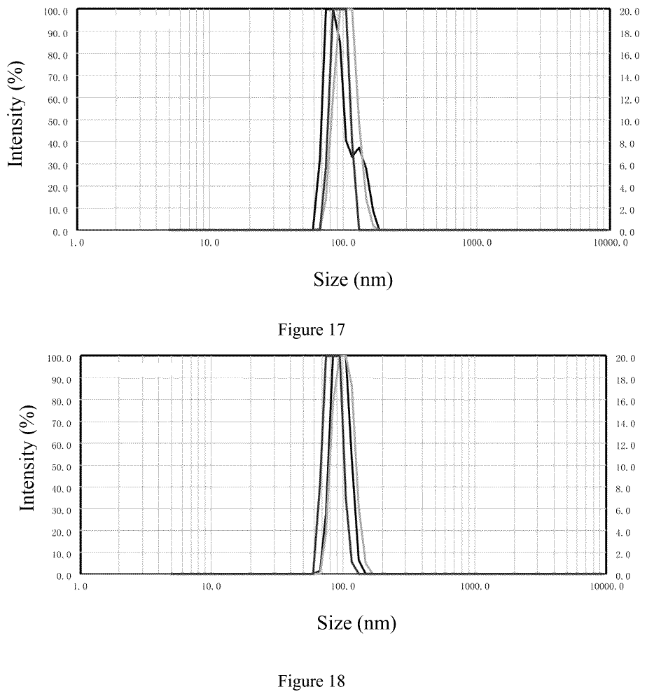

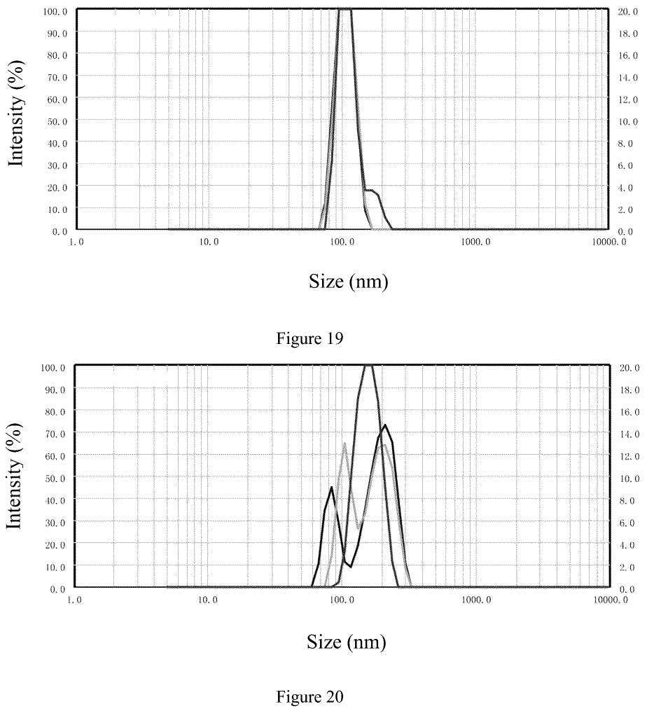

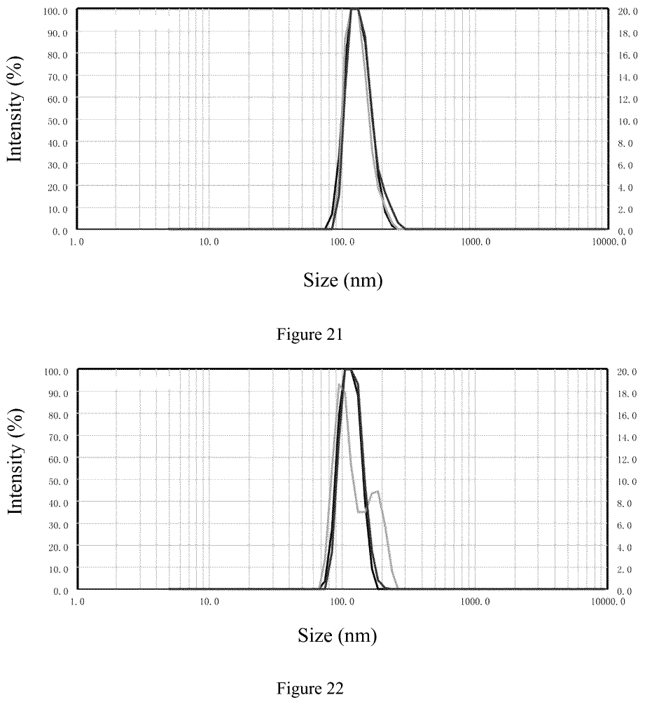

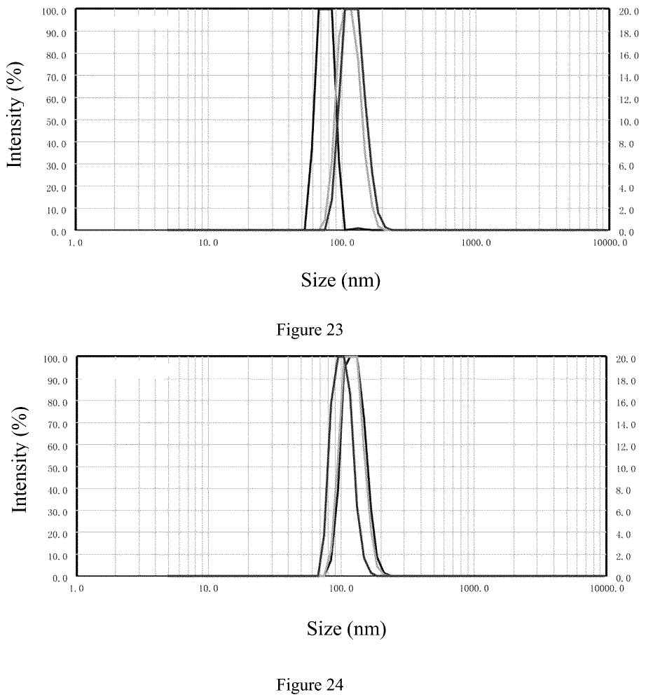

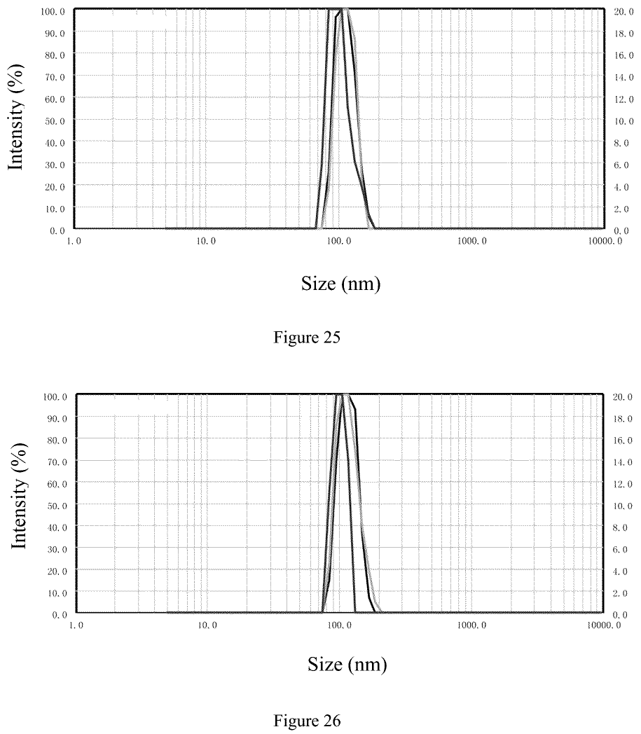

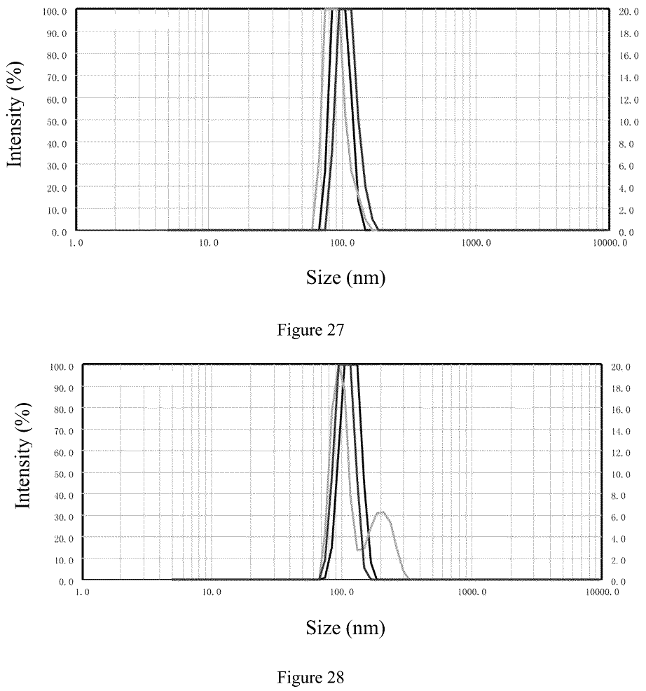

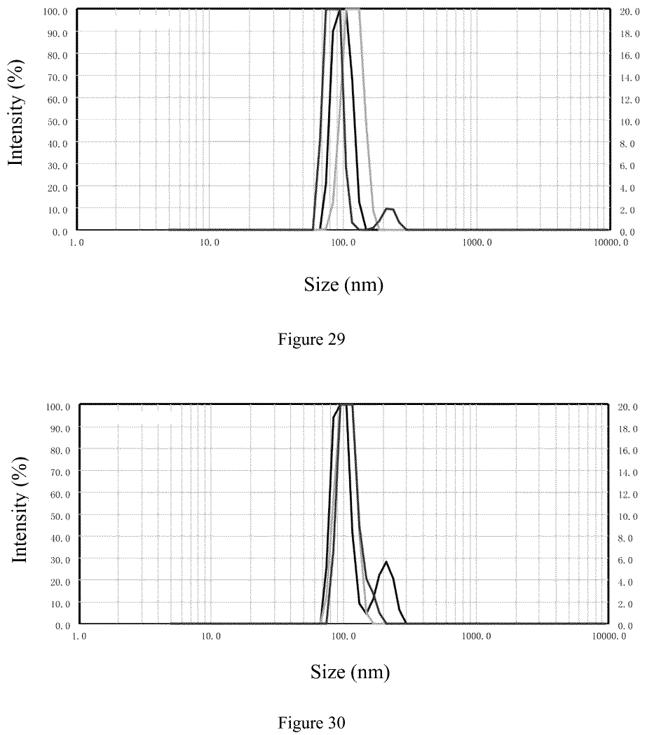

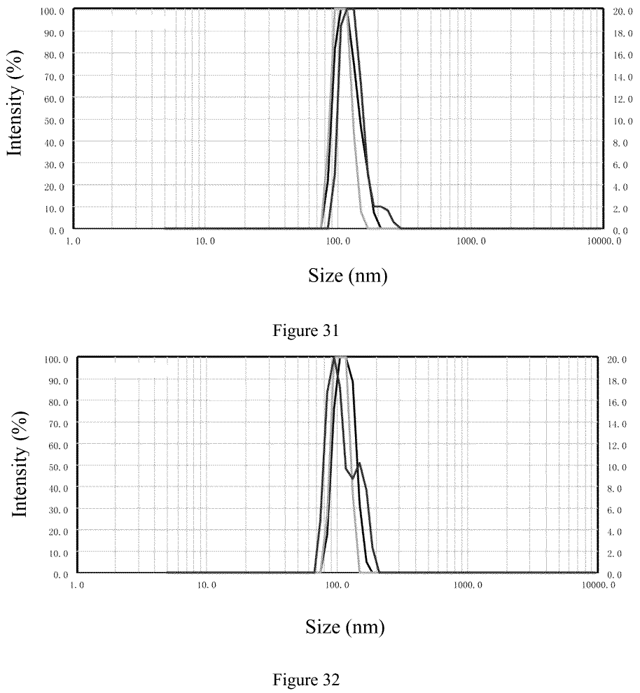

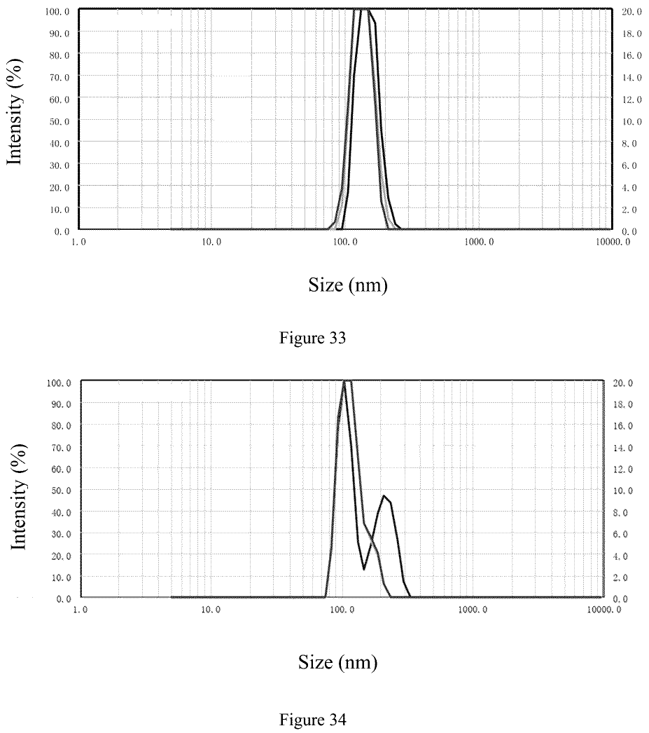

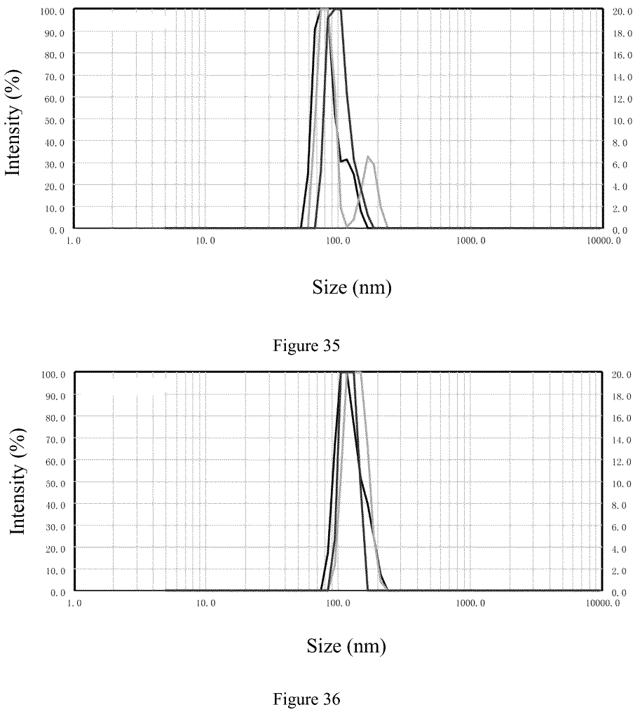

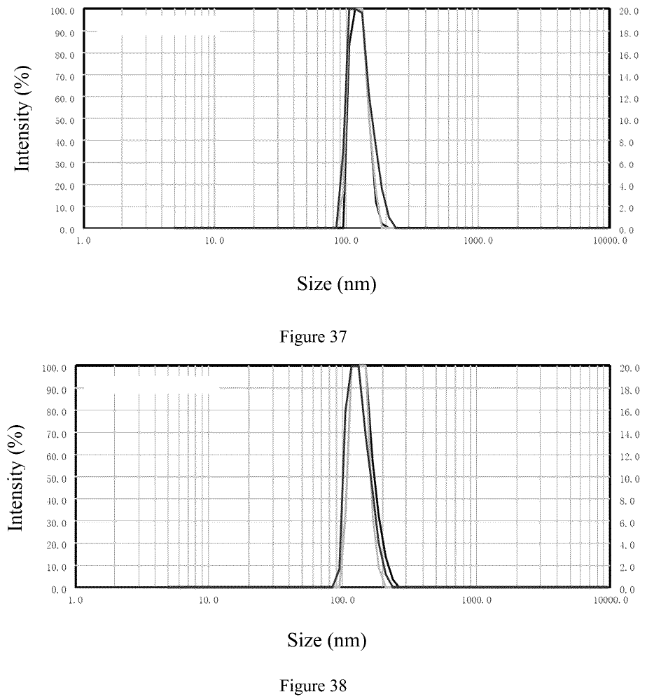

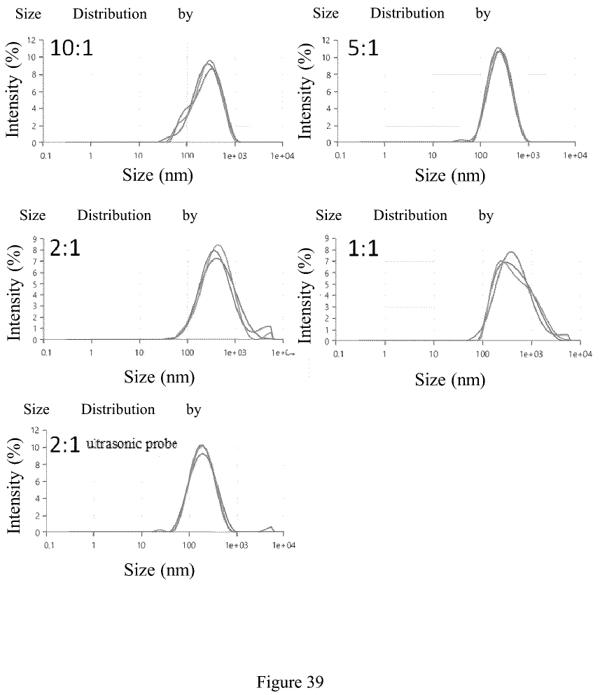

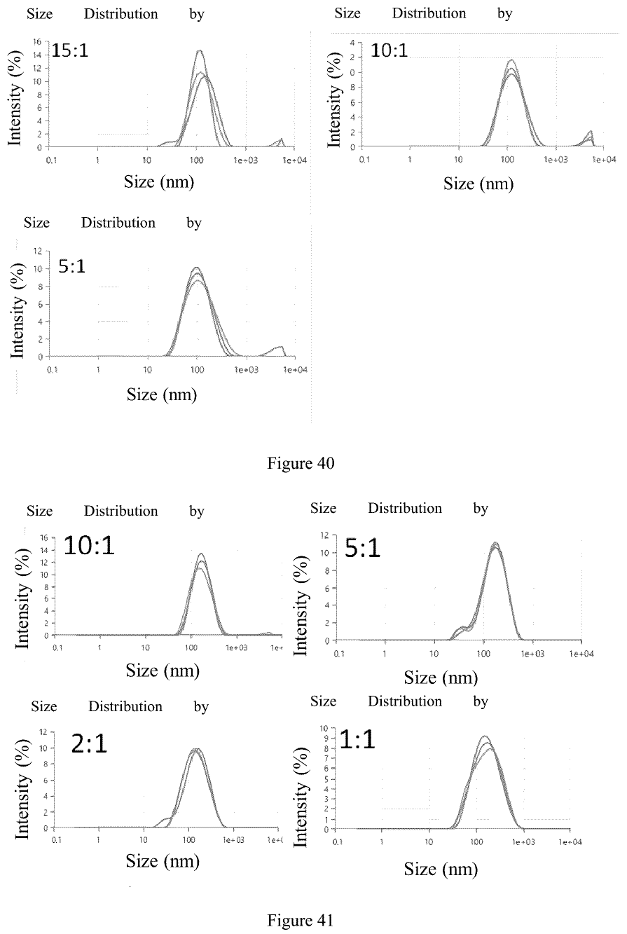

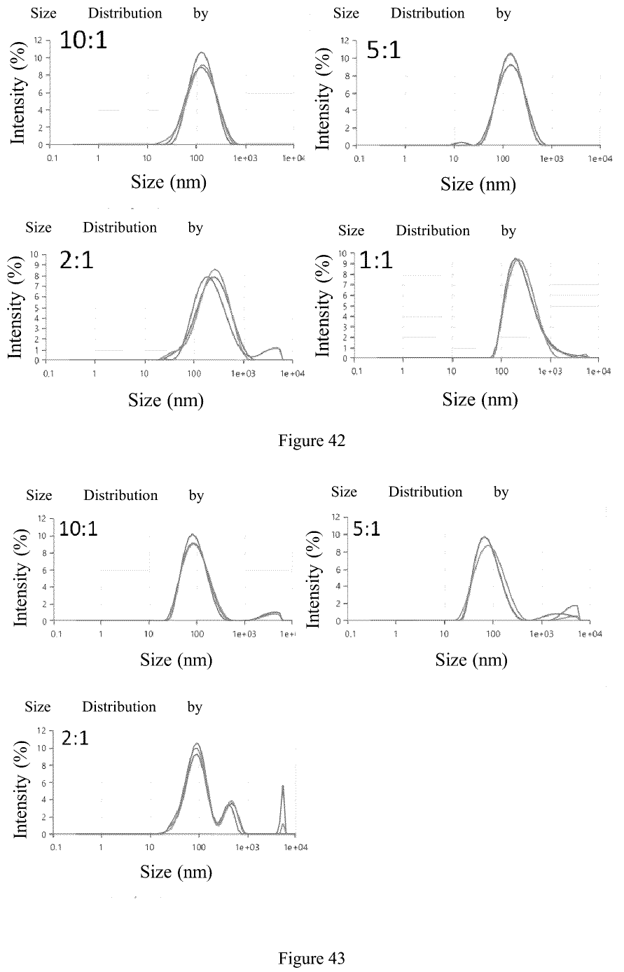

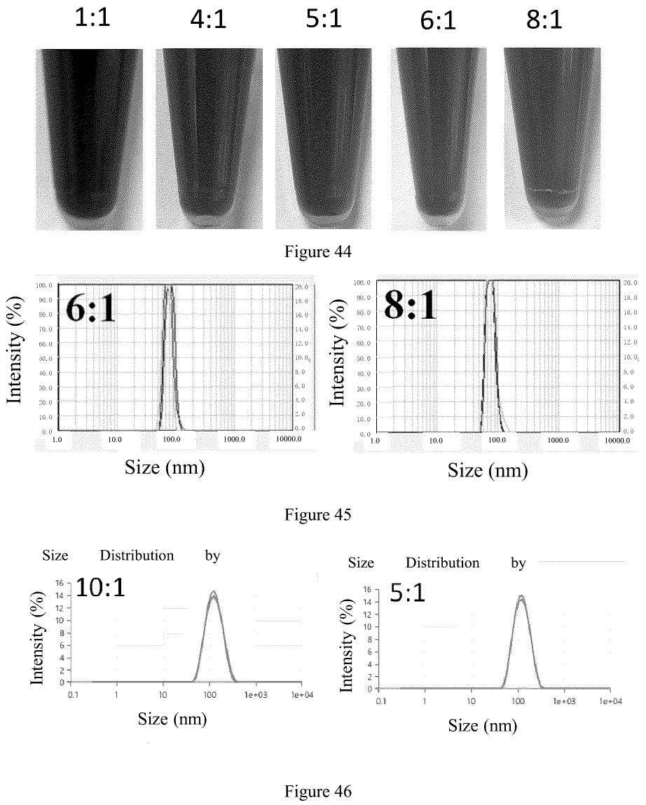

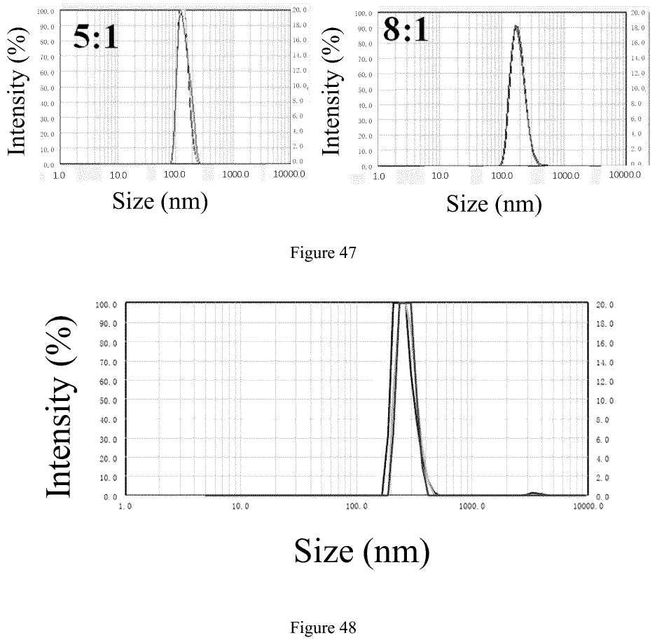

Dynamic light scattering method: The concentration of nanoparticles is 10-100 μg/ml, and the particle size and distribution of nanoparticles are measured with a nanoparticle sizer (laser light source 633 nm). Each sample is measured three times, and the average and variance of the nanoparticle size are calculated. The particle size distribution of different nanoparticles is shown in FIGS. 3 to 43 and FIGS. 45 to 48. The three different curves in the attached figures represent the results of three measurements of the samples.

Since the photosensitizer molecules usually absorb the laser light source (633 nm) of the dynamic light scattering instrument to a certain extent, the intercept in the fitting diagram is low, and there is a certain deviation between the three measurement results of the same sample. Therefore, the average of the three measurements is used to reduce the deviation. The peaks in the micron range in the figure are usually caused by dust.

### Example 4: Determination of Hydrophobic Drug Encapsulation Efficiency

### Example: Determination of SN-38 Encapsulation Efficiency

Take 1 ml of SN-38/ICG solution, filter with a 0.22 μm nylon needle filter, and measure the SN-38 concentration by HPLC.

$$SN - 38 encapsulation effciency \% = \frac{SN 38 concentration after filtration}{SN 38 concentration before filtration} \times 100 \%$$

Unless otherwise specified, the encapsulation efficiency of hydrophobic drugs was measured according to this method.

### Example 5: Preparation of SN-38/ICG Nanoparticles (D₁(S)=0.3 mm, D₃(IN)=5.4 mm, First Phase 13 ml/min, Second Phase 203 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 13 | 203 |

Calculated based on the fluid in the circular pipeline, the combined phase Re=839, with no static mixer.
The first phase outlet flow velocity is: 3.1 m/s
The second phase flow velocity is: 0.15 m/s
FVR=20.8
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 61 ± 4 | 71 ± 5 | 90 ± 15 | 123 ± 18 | 96.0% |

### Example 6: Preparation of SN-38/ICG Nanoparticles (D₁(S)=0.3 mm, D₃(IN)=5.4 mm, First Phase 30 ml/min, Second Phase 468 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.,

| First phase | Second phase |
|---|---|
| ml/min | ml/min |
| 30 | 468 |

Calculated based on the fluid in the circular pipeline, the combined phase Re-1934, with no static mixer.
The first phase outlet flow velocity is: 7.1 m/s
The second phase flow velocity is: 0.34 m/s
FVR=20.8
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 68 ± 6 | 79 ± 8 | 93 ± 11 | 101 ± 6 | 94.3% |

### Example 7: Preparation of SN-38/ICG Nanoparticles (D₁(S)=0.3 mm, D₃(IN)=5.4 mm, First Phase 40 ml/min, Second Phase 624 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 40 | 624 |

Calculated based on the fluid in the circular pipeline, the combined phase Re=2579, with no static mixer.
The first phase outlet flow velocity is: 9.43 m/s
The second phase flow velocity is: 0.45 m/s
FVR=20.8
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 85 ± 5 | 101 ± 5 | 118 ± 7 | 99 ± 5 | 92.0% |

### Example 8: Preparation of SN-38/ICG Nanoparticles (D₁(S)=0.3 mm, D₃(IN)=5.4 mm, First Phase 60 ml/min, Second Phase 936 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 60 | 936 |

Calculated based on the fluid in the circular pipeline, the combined phase Re=3868, with no static mixer.
The first phase outlet flow velocity is: 14.1 m/s
The second phase flow velocity is: 0.68 m/s
FVR=20.8
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 86 ± 5 | 103 ± 6 | 126 ± 10 | 108 ± 9 | 96.0% |

### Example 9: Preparation of SN-38/ICG Nanoparticles (D₁(S)=0.3 mm, D₃(IN)=5.4 mm, First Phase 80 ml/min, Second Phase 1248 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 80 | 1248 |

Calculated based on the fluid in the circular pipeline, the combined phase Re=5158, with no static mixer.
The first phase outlet flow velocity is: 18.9 m/s
The second phase flow velocity is: 0.91 m/s
FVR=20.8
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 88 ± 15 | 109 ± 22 | 138 ± 34 | 99 ± 5 | 97.8% |

### Example 10: Preparation of SN-38/ICG Nanoparticles (D₁(S)=0.3 mm, D₃(IN)=5.4 mm, First Phase 100 ml/min, Second Phase 1560 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1560 |

Calculated based on the fluid in the circular pipeline, the combined phase Re-6477, with no static mixer.
The first phase outlet flow velocity is: 23.6 m/s
The second phase flow velocity is: 1.14 m/s
FVR=20.8
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 76 ± 16 | 89 ± 20 | 107 ± 27 | 104 ± 4 | 95.3% |

### Example 11: Preparation of SN-38/ICG Nanoparticles (D₁(S)=0.3 mm, D₃(IN)=5.4 mm, SK Type Static Mixer, First Phase 13 ml/min, Second Phase 203 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 13 | 203 |

Calculated based on the fluid in the circular pipeline, the combined phase Re=839, with SK type static mixer, static mixer size: 5.3 mm*85 mm, with a total of 16 repeating spiral plates.
The first phase outlet flow velocity is: 3.1 m/s
The second phase flow velocity is: 0.15 m/s
FVR=20.8
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 85 ± 15 | 113 ± 22 | 192 ± 12 | 139 ± 12 | 91.8% |

### Example 12: Preparation of SN-38/ICG Nanoparticles (D₁(S)=0.3 mm, D₃(IN)=5.4 mm, SK Type Static Mixer, First Phase 30 ml/min, Second Phase 468 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 30 | 468 |

Calculated based on the fluid in the circular pipeline, the combined phase Re=1934, with SK type static mixer, static mixer size: 5.3 mm*85 mm, with a total of 16 repeating spiral plates.
The first phase outlet flow velocity is: 7.1 m/s
The second phase flow velocity is: 0.34 m/s
FVR=20.8
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 98 ± 3 | 119 ± 2 | 152 ± 4 | 107 ± 13 | 95.6% |

### Example 13: Preparation of SN-38/ICG Nanoparticles (D₁(S)=0.3 mm, D₃(IN)=5.4 mm, SK Type Static Mixer, First Phase 40 ml/min, Second Phase 624 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 40 | 624 |

Calculated based on the fluid in the circular pipeline, the combined phase Re=2579, with SK type static mixer, static mixer size: 5.3 mm*85 mm, with a total of 16 repeating spiral plates.
The first phase outlet flow velocity is: 9.43 m/s
The second phase flow velocity is: 0.45 m/s
FVR=20.8
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 84 ± 11 | 99 ± 13 | 119 ± 18 | 118 ± 1 | 95.7% |

### Example 14: Preparation of SN-38/ICG Nanoparticles (D₁(S)=0.3 mm, D₃(IN)=5.4 mm, SK Type Static Mixer, First Phase 60 ml/min, Second Phase 936 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 60 | 936 |

Calculated based on the fluid in the circular pipeline, the combined phase Re=3868, with SK type static mixer, static mixer size: 5.3 mm*85 mm, with a total of 16 repeating spiral plates.
The first phase outlet flow velocity is: 14.1 m/s
The second phase flow velocity is: 0.68 m/s
FVR=20.8
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 88 ± 5 | 110 ± 6 | 157 ± 9 | 104 ± 9 | 94.8% |

### Example 15: Preparation of SN-38/ICG Nanoparticles (D₁(S)=0.3 mm, D₃(IN)=5.4 mm, SK Type Static Mixer, First Phase 80 ml/min, Second Phase 1248 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 80 | 1248 |

Calculated based on the fluid in the circular pipeline, the combined phase Re=5158, with SK type static mixer, static mixer size: 5.3 mm*85 mm, with a total of 16 repeating spiral plates.
The first phase outlet flow velocity is: 18.9 m/s
The second phase flow velocity is: 0.91 m/s
FVR=20.8
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 98 ± 3 | 119 ± 2 | 152 ± 4 | 107 ± 13 | 99.7% |

### Example 16: Preparation of SN-38/ICG Nanoparticles (D₁(S)=0.3 mm, D₃(IN)=5.4 mm, SK Type Static Mixer, First Phase 100 ml/min, Second Phase 1560 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1560 |

Calculated based on the fluid in the circular pipeline, the combined phase Re-6477, with SK type static mixer, static mixer size: 5.3 mm*85 mm, with a total of 16 repeating spiral plates.
The first phase outlet flow velocity is: 23.6 m/s
The second phase flow velocity is: 1.14 m/s
FVR=20.8
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 98 ± 3 | 119 ± 2 | 152 ± 4 | 107 ± 13 | 95.9% |

### Example 17: Preparation of SN-38/ICG Nanoparticles (D₁(S)=0.6 mm, D₃(IN)=5.4 mm, First Phase 13 ml/min, Second Phase 203 ml/min)

Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 13 | 203 |

Calculated based on the fluid in the circular pipeline, the combined phase Re=839, with no static mixer.
The first phase outlet flow velocity is: 0.77 m/s
The second phase flow velocity is: 0.15 m/s
FVR=5.2
Production quantity: 15 ml-20 ml

| | | | | Filtration | Filtration |
|---|---|---|---|---|---|
| | | | | encapsula- | encapsula- |
| | | | Average | tion | tion |
| D10 | D50 | D90 | diameter | efficiency | efficiency |
| nm | nm | nm | nm | (SN-38) | (ICG) |
| 123 ± 9 | 269 ± 11 | 457 ± 25 | 222 ± 2 | 29.1% | 85.3% |

### Example 18: Preparation of SN-38/ICG Nanoparticles (D₁(S)=0.6 mm, D₃(IN)=5.4 mm, First Phase 30 ml/min, Second Phase 468 ml/min)

Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 30 | 468 |

Calculated based on the fluid in the circular pipeline, the combined phase Re=1934, with no static mixer.
The first phase outlet flow velocity is: 1.77 m/s
The second phase flow velocity is: 0.34 m/s
FVR=5.2
Production quantity: 15 ml-20 ml

| | | | | Filtration | Filtration |
|---|---|---|---|---|---|
| | | | | encapsula- | encapsula- |
| | | | Average | tion | tion |
| D10 | D50 | D90 | diameter | efficiency | efficiency |
| nm | nm | nm | nm | (SN-38) | (ICG) |
| 130 ± 15 | 199 ± 20 | 303 ± 62 | 161 ± 13 | 61.9% | 91.8% |

### Example 19: Preparation of SN-38/ICG Nanoparticles (D₁(S)=0.6 mm, D₃(IN)=5.4 mm, First Phase 60 ml/min, Second Phase 936 ml/min)

Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 60 | 936 |

Calculated based on the fluid in the circular pipeline, the combined phase Re=3868, with no static mixer.
The first phase outlet flow velocity is: 3.54 m/s
The second phase flow velocity is: 0.68 m/s
FVR=5.2
Production quantity: 15 ml-20 ml

| | | | | Filtration | Filtration |
|---|---|---|---|---|---|
| | | | | encapsula- | encapsula- |
| | | | Average | tion | tion |
| D10 | D50 | D90 | diameter | efficiency | efficiency |
| nm | nm | nm | nm | (SN-38) | (ICG) |
| 79 ± 7 | 96 ± 8 | 124 ± 13 | 98 ± 4 | 98.4% | 98.0% |

### Example 20: Preparation of SN-38/ICG Nanoparticles (D₁(S)=0.6 mm, D₃(IN)=5.4 mm, First Phase 80 ml/min, Second Phase 1248 ml/min)

Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.:

| First phase | Second phase |
|---|---|
| ml/min | ml/min |
| 80 | 1248 |

Calculated based on the fluid in the circular pipeline, the combined phase Re=5158, with no static mixer.
The first phase outlet flow velocity is: 4.72 m/s
The second phase flow velocity is: 0.91 m/s
FVR=5.2
Production quantity: 15 ml-20 ml

| | | | | Filtration | Filtration |
|---|---|---|---|---|---|
| | | | | encapsula- | encapsula- |
| | | | Average | tion | tion |
| D10 | D50 | D90 | diameter | efficiency | efficiency |
| nm | nm | nm | nm | (SN-38) | (ICG) |
| 78 ± 6 | 93 ± 9 | 111 ± 12 | 99 ± 4 | 100.0% | 98.7% |

### Example 21: Preparation of SN-38/ICG Nanoparticles (D₁(S)=0.6 mm, D₃(IN)=5.4 mm, First Phase 100 ml/min, Second Phase 1560 ml/min)

Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1560 |

Calculated based on the fluid in the circular pipeline, the combined phase Re=6477, with no static mixer.
The first phase outlet flow velocity is: 5.89 m/s
The second phase flow velocity is: 1.14 m/s
FVR=5.2
Production quantity: 15 ml-20 ml

| | | | | Filtration | Filtration |
|---|---|---|---|---|---|
| | | | | encapsula- | encapsula- |
| | | | Average | tion | tion |
| D10 | D50 | D90 | diameter | efficiency | efficiency |
| nm | nm | nm | nm | (SN-38) | (ICG) |
| 88 ± 2 | 105 ± 2 | 133 ± 10 | 97 ± 14 | 94.1% | 93.6% |

### Example 22: Preparation of SN-38/ICG Nanoparticles (D₁(S)=0.6 mm, D₃(IN)=5.4 mm, SK Type Static Mixer, First Phase 13 ml/min, Second Phase 203 ml/min)

Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 13 | 203 |

Calculated based on the fluid in the circular pipeline, the combined phase Re-839, with SK type static mixer, static mixer size: 5.3 mm*85 mm, with a total of 16 repeating spiral plates.
The first phase outlet flow velocity is: 0.77 m/s
The second phase flow velocity is: 0.15 m/s
FVR=5.2
Production quantity: 15 ml-20 ml

| | | | | Filtration | Filtration |
|---|---|---|---|---|---|
| | | | | encapsula- | encapsula- |
| | | | Average | tion | tion |
| D10 | D50 | D90 | diameter | efficiency | efficiency |
| nm | nm | nm | nm | (SN-38) | (ICG) |
| 99 ± 20 | 166 ± 11 | 232 ± 26 | 152 ± 2 | 67.4% | 88.8% |

### Example 23: Preparation of SN-38/ICG Nanoparticles (D₁(S)=0.6 mm, D₃(IN)=5.4 mm, SK Type Static Mixer, First Phase 30 ml/min, Second Phase 468 ml/min)

Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 30 | 468 |

Calculated based on the fluid in the circular pipeline, the combined phase Re-1934, with SK type static mixer, static mixer size: 5.3 mm*85 mm, with a total of 16 repeating spiral plates.
The first phase outlet flow velocity is: 1.77 m/s
The second phase flow velocity is: 0.34 m/s
FVR=5.2
Production quantity: 15 ml-20 ml

| | | | | Filtration | Filtration |
|---|---|---|---|---|---|
| | | | Average | encapsulation | encapsulation |
| D10 | D50 | D90 | diameter | efficiency | efficiency |
| nm | nm | nm | nm | (SN-38) | (ICG) |
| 103 ± 2 | 128 ± 4 | 172 ± 7 | 119 ± 8 | 93.4% | 95.3% |

### Example 24: Preparation of SN-38/ICG Nanoparticles (D₁(S)=0.6 mm, D₃(IN)=5.4 mm, SK Type Static Mixer, First Phase 60 ml/min, Second Phase 936 ml/min)

Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 60 | 936 |

Calculated based on the fluid in the circular pipeline, the combined phase Re=3868, with SK type static mixer, static mixer size: 5.3 mm*85 mm, with a total of 16 repeating spiral plates.
The first phase outlet flow velocity is: 3.54 m/s
The second phase flow velocity is: 0.68 m/s
FVR=5.2
Production quantity: 15 ml-20 ml

| | | | | Filtration | Filtration |
|---|---|---|---|---|---|
| | | | | encapsula- | encapsula- |
| | | | Average | tion | tion |
| D10 | D50 | D90 | diameter | efficiency | efficiency |
| nm | nm | nm | nm | (SN-38) | (ICG) |
| 90 ± 4 | 113 ± 2 | 159 ± 29 | 110 ± 3 | 97.5% | 97.6% |

### Example 25: Preparation of SN-38/ICG Nanoparticles (D₁(S)=0.6 mm, D₃(IN)=5.4 mm, SK Type Static Mixer, First Phase 80 ml/min, Second Phase 1248 ml/min)

Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 80 | 1248 |

Calculated based on the fluid in the circular pipeline, the combined phase Re-5158, with SK type static mixer, static mixer size: 5.3 mm*85 mm, with a total of 16 repeating spiral plates.
The first phase outlet flow velocity is: 4.72 m/s
The second phase flow velocity is: 0.91 m/s
FVR=5.2
Production quantity: 15 ml-20 ml

| | | | | Filtration | Filtration |
|---|---|---|---|---|---|
| | | | | encapsula- | encapsula- |
| | | | Average | tion | tion |
| D10 | D50 | D90 | diameter | efficiency | efficiency |
| nm | nm | nm | nm | (SN-38) | (ICG) |
| 83 ± 17 | 101 ± 23 | 126 ± 35 | 109 ± 5 | 98.8% | 98.2% |

### Example 26: Preparation of SN-38/ICG Nanoparticles (D₁(S)=0.6 mm, D₃(IN)=5.4 mm, SK Type Static Mixer, First Phase 100 ml/min, Second Phase 1560 ml/min)

Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1560 |

Calculated based on the fluid in the circular pipeline, the combined phase Re-6477, with SK type static mixer, static mixer size: 5.3 mm*85 mm, with a total of 16 repeating spiral plates.
The first phase outlet flow velocity is: 5.89 m/s
The second phase flow velocity is: 1.14 m/s
FVR=5.2
Production quantity: 15 ml-20 ml

| | | | | Filtration | Filtration |
|---|---|---|---|---|---|
| | | | | encapsula- | encapsula- |
| | | | Average | tion | tion |
| D10 | D50 | D90 | diameter | efficiency | efficiency |
| nm | nm | nm | nm | (SN-38) | (ICG) |
| 93 ± 9 | 113 ± 12 | 143 ± 17 | 111 ± 8 | 98.8% | 100.3% |

### Example 27: Preparation of SN-38/ICG Nanoparticles (D₁(S)=0.3 mm, D₃(IN)=5.4 mm, First Phase 100 ml/min, Second Phase 1248 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1248 |

Calculated based on the fluid in the circular pipeline, the combined phase Re-5236, with no static mixer.
The first phase outlet flow velocity is: 23.6 m/s
The second phase flow velocity is: 0.91 m/s
FVR=26.0
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 89 ± 7 | 106 ± 7 | 133 ± 5 | 109 ± 3 | 95.2% |

### Example 28: Preparation of SN-38/ICG Nanoparticles (D₁(S)=0.3 mm, D₃(IN)=5.4 mm, First Phase 100 ml/min, Second Phase 1326 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1326 |

Calculated based on the fluid in the circular pipeline, the combined phase Re=5539, with no static mixer.
The first phase outlet flow velocity is: 23.6 m/s
The second phase flow velocity is: 0.97 m/s
FVR=24.4
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 92 ± 3 | 108 ± 7 | 132 ± 16 | 112 ± 3 | 96.3% |

### Example 29: Preparation of SN-38/ICG Nanoparticles (D₁(S)=0.3 mm, D₃(IN)=5.4 mm, First Phase 100 ml/min, Second Phase 1404 ml/min)

Spray hole diameter at the end of first pipeline D₁(S)=0.3 mm;
Inner diameter of the second pipeline D₂(IN)=5.4 mm;

Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1404 |

Calculated based on the fluid in the circular pipeline, the combined phase Re=5841, with no static mixer.
The first phase outlet flow velocity is: 23.6 m/s
The second phase flow velocity is: 1.02 m/s
FVR=23.1
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 81 ± 9 | 96 ± 10 | 119 ± 11 | 114 ± 6 | 98.2% |

### Example 30: Preparation of SN-38/ICG Nanoparticles (D₁(S)=0.3 mm, D₃(IN)=5.4 mm, First Phase 100 ml/min, Second Phase 1482 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1482 |

Calculated based on the fluid in the circular pipeline, the combined phase Re=5236, with no static mixer.
The first phase outlet flow velocity is: 23.6 m/s
The second phase flow velocity is: 1.08 m/s
FVR=21.9
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 84 ± 10 | 107 ± 8 | 189 ± 43 | 119 ± 12 | 98.8% |

### Example 31: Preparation of SN-38/ICG Nanoparticles (D₁(S)=0.3 mm, D₃(IN)=5.4 mm, First Phase 100 ml/min, Second Phase 1638 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1638 |

Calculated based on the fluid in the circular pipeline, the combined phase Re=6750, with no static mixer.
The first phase outlet flow velocity is: 23.6 m/s
The second phase flow velocity is: 1.19 m/s
FVR=19.8
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 83 ± 13 | 100 ± 17 | 120 ± 20 | 119 ± 6 | 95.2% |

### Example 32: Preparation of SN-38/ICG Nanoparticles (D₁(S)=0.3 mm, D₃(IN)=5.4 mm, First Phase 100 ml/min, Second Phase 1716 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1716 |

Calculated based on the fluid in the circular pipeline, the combined phase Re=7053, with no static mixer.
The first phase outlet flow velocity is: 23.6 m/s
The second phase flow velocity is: 1.25 m/s
FVR=18.9
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 86 ± 4 | 103 ± 4 | 154 ± 42 | 105 ± 5 | 96.2% |

### Example 33: Preparation of SN-38/ICG Nanoparticles (D₁(S)=0.3 mm, D₃(IN)=5.4 mm, First Phase 100 ml/min, Second Phase 1794 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1794 |

Calculated based on the fluid in the circular pipeline, the combined phase Re=7356, with no static mixer.
The first phase outlet flow velocity is: 23.6 m/s
The second phase flow velocity is: 1.31 m/s
FVR=18.1
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 95 ± 6 | 114 ± 10 | 146 ± 19 | 117 ± 10 | 96.5% |

### Example 34: Preparation of SN-38/ICG Nanoparticles (D₁(S)=0.3 mm, D₃(IN)=5.4 mm, First Phase 100 ml/min, Second Phase 1872 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1872 |

Calculated based on the fluid in the circular pipeline, the combined phase Re=7659, with no static mixer.
The first phase outlet flow velocity is: 23.6 m/s
The second phase flow velocity is: 1.36 m/s
FVR=17.3
Production quantity: 15 ml-20 ml

| | | | | Filtration |
|---|---|---|---|---|
| | | | Average | encapsulation |
| D10 | D50 | D90 | diameter | efficiency |
| nm | nm | nm | nm | (SN-38) |
| 89 ± 6 | 107 ± 5 | 139 ± 17 | 114 ± 5 | 96.3% |

### Example 35: Preparation of SN-38/ICG Nanoparticles (D₁(S)=0.3 mm, D₃(IN)=5.4 mm, First Phase 100 ml/min, Second Phase 1404 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1404 |

Calculated based on the fluid in the circular pipeline, the combined phase Re=5841, with no static mixer.
The first phase outlet flow velocity is: 23.6 m/s
The second phase flow velocity is: 1.02 m/s
FVR=23.1
Production quantity: 60 ml

| | | | | Filtration |
|---|---|---|---|---|
| | | | Average | encapsulation |
| D10 | D50 | D90 | diameter | efficiency |
| nm | nm | nm | nm | (SN-38) |
| 110 ± 6 | 137 ± 7 | 172 ± 9 | 127 ± 6 | 91.7% |

### Example 36: Preparation of SN-38/ICG Nanoparticles (D₁(S)=0.3 mm, D₃(IN)=5.4 mm, First Phase 100 ml/min, Second Phase 1404 ml/min)

Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1404 |

Calculated based on the fluid in the circular pipeline, the combined phase Re=5841, with no static mixer.
The first phase outlet flow velocity is: 23.6 m/s
The second phase flow velocity is: 1.02 m/s
FVR=23.1
Production quantity: 557 ml

| | | | | Filtration |
|---|---|---|---|---|
| | | | Average | encapsulation |
| D10 | D50 | D90 | diameter | efficiency |
| nm | nm | nm | nm | (SN-38) |
| 92.5 ± 0.1 | 114 ± 4 | 186 ± 46 | 129 ± 10 | 89.2% |

### Example 37: Preparation of SN-38/ICG Nanoparticles (D₁(S)=0.6 mm, D₃(IN)=5.4 mm, First Phase 100 ml/min, Second Phase 1560 ml/min)

Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1560 |

Calculated based on the fluid in the circular pipeline, the combined phase Re-6477, with no static mixer.
The first phase outlet flow velocity is: 5.89 m/s
The second phase flow velocity is: 1.14 m/s
FVR=5.2
Production quantity: 60 ml

| | | | | Filtration |
|---|---|---|---|---|
| | | | Average | encapsulation |
| D10 | D50 | D90 | diameter | efficiency |
| nm | nm | nm | nm | (SN-38) |
| 71 ± 5 | 96 ± 20 | 181 ± 16 | 122 ± 5 | 94.2% |

### Example 38: Preparation of SN-38/ICG Nanoparticles (D₁(S)=0.6 mm, D₃(IN)=5.4 mm, SK Type Static Mixer, First Phase 100 ml/min, Second Phase 1560 ml/min)

Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1560 |

Calculated based on the fluid in the circular pipeline, the combined phase Re=6477, with SK type static mixer, static mixer size: 5.3 mm*85 mm, with a total of 16 repeating spiral plates.
The first phase outlet flow velocity is: 5.89 m/s
The second phase flow velocity is: 1.14 m/s
FVR=5.2
Production quantity: 60 ml

| | | | | Filtration |
|---|---|---|---|---|
| | | | Average | encapsulation |
| D10 | D50 | D90 | diameter | efficiency |
| nm | nm | nm | nm | (SN-38) |
| 87 ± 14 | 115 ± 7 | 164 ± 13 | 114 ± 2 | 94.8% |

### Example 39: Preparation of SN-38/ICG Nanoparticles (D₁(S)=0.6 mm, D₃(IN)=5.4 mm, SK Type Static Mixer, First Phase 80 ml/min, Second Phase 1248 ml/min)

Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 80 | 1248 |

Calculated based on the fluid in the circular pipeline, the combined phase Re=5158, with SK type static mixer, static mixer size: 5.3 mm*85 mm, with a total of 16 repeating spiral plates.
The first phase outlet flow velocity is: 4.72 m/s
The second phase flow velocity is: 0.91 m/s
FVR=5.2
Production quantity: 425 ml

The SN-38 concentration of the preparation solution was 2.05 mg/ml, the SN-38 concentration after filtration of 0.22 μm membrane was 1.89 mg/ml, the encapsulation efficiency of SN-38: 92.2%, the original solution was added 871.4 g water and purified with 0.05 m²30 kDa PES ultrafiltration membrane.

Ultrafiltrate was concentrated to 166.1 g, SN-38 concentration was 5.07 mg/ml; After filtration with 0.22 μm membrane, the SN-38 concentration was 4.60 mg/ml, and the encapsulation efficiency of SN-38 after ultrafiltration was 90.8%.

Particle size change before and after ultrafiltration:

| | | | | Average | |
|---|---|---|---|---|---|
| | D10 | D50 | D90 | diameter | |
| | nm | nm | nm | nm | PDI |
| Before | 103 ± 3 | 121 ± 1 | 150 ± 10 | 126 ± 4 | 0.11 ± 0.1 |
| ultrafiltration | | | | | |
| After | 110 ± 4 | 131 ± 5 | 166 ± 10 | 132 ± 2 | 0.19 ± 0.08 |
| ultrafiltration | | | | | |

### Example 40: Preparation of SN-38/ICG Nanoparticles (SN-38:ICG=15:1-5:1, Molar Ratio)

SN-38 was dissolved in DMSO (39.2 mg/ml, 0.1 M), ICG was dissolved in DMSO (77.5 mg/ml, 0.1 M), SN-38 in DMSO and ICG in DMSO were mixed in proportion following the table below as the first phase solution, and water as the second phase solution.
Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1560 |

Calculated based on the fluid in the circular pipeline, the combined phase Re-6477, with no static mixer.
The first phase outlet flow velocity is: 5.89 m/s
The second phase flow velocity is: 1.14 m/s
FVR=5.2
Production quantity: 15 ml-20 ml

| | | | | | | | SN-38 | ICG |
|---|---|---|---|---|---|---|---|---|
| SN38/ICG | | | | Average | | | encapsulation | encapsulation |
| Molar | D10 | D50 | D90 | diameter | C_{SN-38} | C_{ICG} | efficiency | efficiency |
| ratio | nm | nm | nm | nm | mg/ml | mg/ml | % | % |
| 15:1 | 63 | 127 | 232 | 139 | 2.08 | 0.23 | 96.1 | 95.6 |
| 10:1 | 65 | 128 | 279 | 117 | 2.06 | 0.39 | 96.6 | 97.4 |
| 5:1 | 50 | 108 | 254 | 94 | 2.06 | 0.80 | 98 | 98.7 |

### Example 41: Preparation of Camptothecine/ICG Nanoparticles (Camptothecine:ICG=10:1-1:1, Molar Ratio)

Camptothecin was dissolved in DMSO (20 mg/ml, 0.057 M), ICG was dissolved in DMSO (77.5 mg/ml, 0.1 M), camptothecin in DMSO and ICG in DMSO were mixed in proportion following the table below as the first phase solution, and water as the second phase solution.
Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1560 |

Calculated based on the fluid in the circular pipeline, the combined phase Re=6477, with no static mixer.
The first phase outlet flow velocity is: 5.89 m/s
The second phase flow velocity is: 1.14 m/s
FVR=5.2
Production quantity: 15 ml-20 ml

| | | | | Average | | CPT encapsulation |
|---|---|---|---|---|---|---|
| Camptothecin:ICG | D10 | D50 | D90 | diameter | | efficiency |
| Molar ratio | nm | nm | nm | nm | PDI | % |
| 10:1 | 54.82 | 76.95 | 126.4 | 143.9 | 0.22 | 87 |
| 5:1 | 21.31 | 28 | 40.41 | 133.9 | 0.25 | 88 |
| 2:1 | 27.39 | 37.75 | 58.84 | 119.5 | 0.25 | 88 |
| 1:1 | 33.3 | 45.59 | 71.03 | 136.7 | 0.27 | 85 |

### Example 42: Preparation of 10-Hydroxycamptothecine/ICG Nanoparticles (10-Hydroxycamptothecine:ICG=10:1-1:1, Molar Ratio)

10-Hydroxycamptothecin was dissolved in DMSO (36.4 mg/ml, 0.1 M), ICG was dissolved in DMSO (77.5 mg/ml, 0.1 M), 10-Hydroxycamptothecin in DMSO and ICG in DMSO were mixed in proportion following the table below as the first phase solution, and water as the second phase solution.
Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1560 |

Calculated based on the fluid in the circular pipeline, the combined phase Re-6477, with no static mixer.
The first phase outlet flow velocity is: 5.89 m/s
The second phase flow velocity is: 1.14 m/s
FVR=5.2
Production quantity: 15 ml-20 ml

| | | | | | | 10-Hydroxycamptoth | ICG |
|---|---|---|---|---|---|---|---|
| 10- | | | | Average | | encapsulation | encapsulation |
| Hydroxycamptothecin:ICG | D10 | D50 | D90 | diameter | | efficiency | efficiency |
| Molar ratio | nm | nm | nm | nm | PDI | % | % |
| 10:1 | 23.46 | 32.57 | 51.99 | 103 | 0.29 | 97 | 100 |
| 5:1 | 27.52 | 37.7 | 59.34 | 120.4 | 0.25 | 93 | 95 |
| 2:1 | 19.86 | 26.44 | 44.53 | 190.3 | 0.34 | 93 | 98 |
| 1:1 | 74.56 | 104.6 | 189.4 | 226.3 | 0.25 | 85 | 97 |

### Example 43: Preparation of Exatecan/ICG Nanoparticles (Exatecan:ICG=1:1-10:1, Molar Ratio)

Exatecan (free base, Mw=435.4) was dissolved in DMSO (43.5 mg/ml, 0.1 M), ICG was dissolved in DMSO (77.5 mg/ml, 0.1 M), Exatecan in DMSO and ICG in DMSO were mixed in proportion following the table below as the first phase solution, and water as the second phase solution.
Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1560 |

Calculated based on the fluid in the circular pipeline, the combined phase Re-6477, with no static mixer.
The first phase outlet flow velocity is: 5.89 m/s
The second phase flow velocity is: 1.14 m/s
FVR=5.2
Production quantity: 15 ml-20 ml

| | | | | | | Exatecan | ICG |
|---|---|---|---|---|---|---|---|
| | | | | Average | | encapsulation | encapsulation |
| Exatecan:ICG | D10 | D50 | D90 | diameter | | efficiency | efficiency |
| Molar ratio | nm | nm | nm | nm | PDI | % | % |
| 10:1 | 45 | 95 | 254 | 87 | 0.3 | 75 | 74 |
| 5:1 | 39 | 82 | 258 | 76 | 0.32 | 102 | 94 |
| 2:1 | 47 | 101 | 489 | 320 | 0.51 | 101 | 96 |
| 1:1 | — | — | — | — | — | 100 | 95 |

Note:
Exatecan:ICG = 1:1 gave a transparent solution.

### Example 44: Preparation of Dxd/ICG Nanoparticles (Dxd:ICG=10:1-1:1, Molar Ratio)

Dxd (Mw=493.5) was dissolved in DMSO (49.4 mg/ml, 0.1 M), ICG was dissolved in DMSO (77.5 mg/ml, 0.1 M), Dxd in DMSO and ICG in DMSO were mixed in proportion following the table below as the first phase solution, and water as the second phase solution.
Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 100 | 1560 |

Calculated based on the fluid in the circular pipeline, the combined phase Re=6477, with no static mixer.
The first phase outlet flow velocity is: 5.89 m/s
The second phase flow velocity is: 1.14 m/s
FVR=5.2
Production quantity: 15 ml-20 ml

| | | | | | | Dxd | ICG |
|---|---|---|---|---|---|---|---|
| | | | | Average | | encapsulation | encapsulation |
| Dxd:ICG | D10 | D50 | D90 | diameter | | efficiency | efficiency |
| Molar ratio | nm | nm | nm | nm | PDI | % | % |
| 10:1 | 104 | 130 | 165 | 119 | 0.38 | 94 | 99 |
| 5:1 | 120 | 154 | 202 | 123 | 0.24 | 91 | 96 |
| 2:1 | — | — | — | — | — | 104 | 96 |
| 1:1 | — | — | — | — | — | 103 | 95 |

Note:
Dxd:ICG = 2:1-1:1 gave a transparent solution.

### Example 45: Preparation of Sorafenib/ICG Nanoparticles (Sorafenib:ICG=1:1-8:1,Molar Ratio)

Sorafenib was dissolved in DMSO (46.5 mg/ml, 0.1 M), ICG was dissolved in DMSO (77.5 mg/ml, 0.1 M), Sorafenib in DMSO and ICG in DMSO were mixed in proportion following the table below as the first phase solution, and water as the second phase solution.
Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 40 | 400 |

Production quantity: 15 ml-20 ml
After centrifugation, Sorafenib:ICG=1:1 gave a transparent solution, and the precipitate gradually increases when the amount of sorafenib is increased.

| Sorafenib/ICG | Average diameter | | Encapsulation efficiency |
|---|---|---|---|
| Molar ratio | nm | PDI | % |
| 1:1 | Not detected | / | 99.4 |
| 4:1 | Not detected | / | 99.2 |
| 5:1 | Not detected | / | 99.8 |
| 6:1 | 76.7 | 0.098 | 98.2 |
| 8:1 | 76.5 | 0.097 | 96.7 |

Conclusion: When Sorafenib:ICG = 6:1 and 8:1, the particle size can be detected, indicating that sorafenib and ICG can self-assemble into nanoparticles.

### Example 46: Preparation of PTX/ICG Nanoparticles (PTX:ICG-1:1-10:1, Molar Ratio)

PTX was dissolved in MeOH (42.7 mg/ml, 0.05 M), ICG was dissolved in DMSO (77.5 mg/ml, 0.1 M), PTX in MeOH and ICG in DMSO were mixed in proportion following the table below as the first phase solution, and water as the second phase solution.
Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 40 | 400 |

Production quantity: 15 ml-20 ml

| | | | | | | PTX | ICG |
|---|---|---|---|---|---|---|---|
| PTX:ICG | | | | Average | | encapsulation | encapsulation |
| Molar | D10 | D50 | D90 | diameter | | efficiency | efficiency |
| ratio | nm | nm | nm | nm | PDI | % | % |
| 10:1 | 49.24 | 66.96 | 102.9 | 116.6 | 0.13 | 96 | 100 |
| 5:1 | 50.23 | 67.99 | 103.7 | 113.9 | 0.1 | 93 | 94 |
| 2:1 | 0.55 | 0.68 | 0.9 | 6.25 | 0.56 | 98 | 98 |
| 1:1 | 0.56 | 0.68 | 0.87 | 6.09 | 0.51 | 98 | 100 |

### Example 47: Preparation of Curcumin/ICG Nanoparticles (Curcumin:ICG=2:1-8:1, Molar Ratio)

Curcumin was dissolved in DMSO (36.8 mg/ml, 0.1 M), ICG was dissolved in DMSO (77.5 mg/ml, 0.1 M), Curcumin in DMSO and ICG in DMSO were mixed in proportion following the table below as the first phase solution, and water as the second phase solution.
Spray hole diameter at the end of first pipeline D₁(S)=0.3 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 40 | 400 |

Production quantity: 15 ml-20 ml

After centrifugation, the solution with a volume ratio of 2:1 and 4:1 was clear, and no precipitate was found. The precipitation gradually increased from 5:1.

| | Average | | Encapsulation |
|---|---|---|---|
| Curcumin/ICG | diameter | | efficiency |
| Molar ratio | nm | PDI | % |
| 2:1 | Not detected | / | 99.5 |
| 4:1 | Not detected | / | 97.6 |
| 5:1 | 110 | 0.16 | 96.5 |
| 6:1 | 155 | 0.20 | 94.4 |
| 7:1 | 131 | 0.15 | 93.6 |
| 8:1 | 141 | 0.08 | 88.3 |

Note:
when Curcumin:ICG = 2:1-4:1, the particle size is too small and cannot be detected. The particle size gradually increases as the proportion of curcumin increases.

### Example 48: Preparation of SN-38/Ce6 Nanoparticles (SN-38/Ce6=2:1, Molar Ratio)

Ce6 was dissolved in DMSO (59.7 mg/ml, 0.1 M), SN-38 was dissolved in DMSO (39.2 mg/ml, 0.1 M), SN-38 in DMSO and Ce6 in DMSO were mixed in 2:1 volume ratio as the first phase solution, and water as the second phase solution.
Spray hole diameter at the end of first pipeline D₁(S)=0.6 mm;
Inner diameter of the second pipeline D₂(IN)=5.4 mm;
Inner diameter of the combined pipeline D₃(IN)=5.4 mm;
Outer diameter at the end of first pipeline D₁(O)=2 mm;
Outer diameter of the second pipeline D₂(O)=6 mm;
Outer diameter of the combined pipeline D₃(O)=6 mm;
Combined phase length=90 mm;
First phase solution temperature T₁=25° C.;
Second phase solution temperature T₂=25° C.;

| First phase | Second phase |
|---|---|
| ml/min | ml/min |
| 30 | 468 |

Calculated based on the fluid in the circular pipeline, the combined phase Re=1934, with no static mixer.
The first phase outlet flow velocity is: 1.77 m/s
The second phase flow velocity is: 0.34 m/s
FVR=5.2
Production quantity: 15 ml-20 ml

| | | | | Centrifugal |
|---|---|---|---|---|
| | | | Average | encapsulation |
| D10 | D50 | D90 | diameter | efficiency |
| nm | nm | nm | nm | (SN-38) |
| 215 | 258 | 332 | 258 | 93% |

### Comparative Example 1: Preparation of PTX/ICG Nanoparticles by Thin-film Hydration Method

15.72 mg paclitaxel and 36.42 mg ICG were added to a 50 ml eggplant flask and dissolved in 5 ml methanol. The paclitaxel content was −30 wt. %. The methanol was removed in vacuo. Adding 7.860 ml of deionized water pre-warmed at 60° C. and rotating hydration at 60° C. did not yield uniform PTX/ICG nanoparticles.

The published literature shows that mPEG₂ₖ-PLA₂ₖ and PTX can form PTX/mPEG-PLA micelles at −20 nm by thin-film hydration method with a drug load of 30 wt. % and an encapsulation efficiency of >90%.

Therefore, the properties of polymers and photosensitizers are fundamentally different and cannot be simply replaced.

### Comparative Example 2: Preparation of SN-38/ICG Nanoparticles (SN-38:ICG=10:1-5:1, Molar Ratio) by Nanoprecipitation Method in Intermittent Small Batches

SN-38 was dissolved in DMSO (39.2 mg/ml, 0.1 M), ICG was dissolved in DMSO (77.5 mg/ml, 0.1 M), SN-38 in DMSO and ICG in DMSO were filtered with 0.22 μm membrane and mixed in proportion following the table below in 1.5 ml EP tube.

Water was pre-added to 8 ml vials according to the following table, placed in a 120 W ultrasonic bath, SN-38 and ICG DMSO mixture was quickly added to the water with a pipette, and continued to sonicate for 30 s to determine the particle size distribution and encapsulation efficiency.

| | | | | Added | Added | |
|---|---|---|---|---|---|---|
| | | Theoretical | Theoretical | SN-38 | ICG | |
| SN-38:ICG | SN-38:ICG | SN-38 | ICG | DMSO | DMSO | |
| (Molar | (Mass | added | added | solution | solution | H₂O |
| ratio) | ratio) | (mg) | (mg) | (μl) | (μl) | (μl) |
| 10:1 | 5.06 | 2 | 0.39 | 50 | 4.9 | 3945 |
| 5:1 | 2.53 | 2 | 0.79 | 50 | 9.9 | 3940 |
| 2:1 | 1.01 | 2 | 1.97 | 50 | 24.7 | 3925 |
| 1:1 | 0.51 | 2 | 3.95 | 50 | 49.4 | 3901 |

| | | | | | | | | SN-38 | ICG |
|---|---|---|---|---|---|---|---|---|---|
| SN-38/ICG | | | | Average | | | | encapsulation | encapsulation |
| (Molar | D10 | D50 | D90 | diameter | C_{SN-38} | C_{ICG} | | efficiency | efficiency |
| ratio) | (nm) | (nm) | (nm) | (nm) | (mg/ml) | (mg/ml) | PDI | (%) | (%) |
| 10:1 | 36.72 | 49.18 | 74.07 | 207.60 | 0.47 | 0.097 | 0.30 | 72.3 | 89.7 |
| 5:1 | 60.31 | 86.47 | 160.00 | 225.70 | 0.45 | 0.167 | 0.21 | 53.3 | 83.8 |
| 2:1 | 43.99 | 61.48 | 127.60 | 336.90 | 0.46 | 0.44 | 0.36 | 36.9 | 97.7 |
| 1:1 | 80.00 | 117.10 | 232.90 | 341.50 | 0.48 | 0.91 | 0.36 | 27.1 | 97.8 |
| 2:1 | 34.72 | 48.48 | 78.82 | 166.40 | 0.48 | 0.91 | 0.23 | 76.1 | 97.7 |
| ultrasonic | | | | | | | | | |
| probe | | | | | | | | | |

Note:
The 2:1 ultrasonic probe refers to SN-38:ICG = 2:1 (molar ratio) samples continue to be sonicated with a 300 W probe sonicator for 30 s, sonicated for 1 s and stopped for 1 s.

When the molar ratio of SN-38 to ICG was 10:1-1:1, the encapsulation efficiency of SN-38 decreased from 72.3% to 27.1%, and the encapsulation efficiency of SN-38 was only increased to 76.1% from 36.9% with 300 W probe sonicator (SN-38:ICG=2:1).

Although the specific embodiments of the present invention have been described above, those skilled in the art should understand that these are only illustrations, and that various changes or modifications may be made to these embodiments without deviating from the principle and substance of the present invention. Therefore, the scope of protection of the present invention is limited by the attached claims.

1. A production system, which includes (1) a first pipeline, (2) a second pipeline, (3) a combined pipeline and their outlets;
wherein, the first pipeline and the second pipeline are connected to the combined pipeline, the first phase solution enters the combined pipeline through the first pipeline outlet, the second phase solution enters the combined pipeline through the second pipeline outlet, the first phase solution and the second phase solution are mixed in the combined pipeline to form a combined phase; the combined phase flows out through the outlet of the combined pipeline;
the first pipeline outlet is a spray hole with a certain shape and diameter, and the first phase solution passes through the first pipeline and enters the combined pipeline through the spray hole; The spray hole diameter D₁(S) is 0.3-0.6 mm; The combined pipeline inner diameter D₃(IN) is 5.4-50.0 mm.

2. The system of claim 1, wherein the core part of the production system includes: (1) a first pipeline; (2) a second pipeline; (3) a combined pipeline; (4) turbulent mixing device; (5) fluid outlet;
wherein, the first pipeline and the second pipeline are connected to the combined pipeline, the first phase solution enters the combined pipeline through the first pipeline outlet, the second phase solution enters the combined pipeline through the second pipeline outlet, the first phase solution and the second phase solution are mixed in the combined pipeline to form a combined phase. The combined phase is fully mixed by a turbulent mixing device. After the mixing, the composite nanoparticles are collected into a suitable container through the outlet of the combined pipeline.

3. The system of claim 1, wherein the production system fulfills one or more of the following:
(1) the first pipeline is coaxial with the combined pipeline and the second pipeline is perpendicular to the combined pipeline;
(2) the mixing is turbulent mixing; The turbulent mixing can be achieved by adding a turbulent mixing device in the combined pipeline;
(3) the first pipeline outlet is positioned within the combined pipeline.

4. The system of claim 3, wherein the production system fulfills one or more of the following:
(1) the second pipeline inner diameter D₂(IN) is selected from 0.3-50.0 mm;
(2) the combined pipeline length is selected from 6 to 120 cm;
(3) the ratio of the combined pipeline length to the combined pipeline inner diameter is (16-17):1;
(4) the second pipeline inner diameter D₂(IN) is the same as the combined pipeline inner diameter D₃(IN);
(5) the ratio of the spray hole diameter D₁(S) to the combined pipeline inner diameter D₃(IN) is 1:(2-50);
(6) the turbulent mixing achieved by turbulent mixing device includes but not limited to: increasing the fluid flow velocity, changing the degree of pipeline twisting, or adding baffles or special-shaped objects within the pipeline.

5. The system of claim 4, wherein the production system fulfills one or more of the following:
(1) the second pipeline inner diameter D₂(IN) is 5.4 mm;
(2) the combined pipeline inner diameter D₃(IN) is 5.4 mm;
(3) the combined pipeline length is 9 cm;
(4) the ratio of the combined pipeline length to the combined pipeline inner diameter is 16.7:1;
(5) the ratio of the spray hole diameter D₁(S) to the combined pipeline inner diameter D₃(IN) is 1:9 or 1:18;
(6) the first pipeline outer diameter D₁(O) is 2 mm;
(7) the second pipeline outer diameter D₂(O) is 6 mm;
(8) the combined pipeline outer diameter D₃(O) is 6 mm;
(9) the turbulent mixing device is static mixer, and the Reynolds number calculated based on the fluid in the circular tube is selected from 500-100000;
(10) the amount of turbulent mixing device can be one or more;
(11) the materials used in the first pipeline, the second pipeline, the combined pipeline, the turbulent mixing device, and the fluid outlet are each selected from one or more of stainless steel, polytetrafluoroethylene, polyethylene, polypropylene, latex, silicone, or other polymer materials.

6. A production method for producing photosensitizer/anti-tumor drug composite nanoparticles, which includes the following steps: in the production system of claim 1, the first phase solution and the second phase solution are mixed, and the photosensitizer/anti-tumor drug composite nanoparticles are collected from combined phase through the fluid outlet;
the solvent in the first phase solution is a good solvent for an anti-tumor drug or its pharmaceutically acceptable salt, and the solute is (1) an anti-tumor drug or its pharmaceutically acceptable salt and a photosensitizer, or (2) an anti-tumor drug or its pharmaceutically acceptable salt;
the solvent in the second phase solution is an anti-solvent of the anti-tumor drug or its pharmaceutically acceptable salt, and the solute is (1) absent, or (2) a photosensitizer;
when the solute in the first phase solution is an anti-tumor drug or its pharmaceutically acceptable salt and a photosensitizer, the solute in the second phase solution does not exist;
when the solute in the first phase solution is an anti-tumor drug or its pharmaceutically acceptable salt, the solute in the second phase solution is a photosensitizer.

7. The method of claim 6, wherein the production method is any of the following:
method I: (1a). one or more of the anti-tumor drugs or their pharmaceutically acceptable salts and one or more of the photosensitizers are dissolved in a first phase solvent to form a first phase solution, and the first phase solution is a good solvent for the anti-tumor drug or its pharmaceutically acceptable salt and the photosensitizer;
(1b). the second phase solution is an anti-solvent for anti-tumor drugs or their pharmaceutically acceptable salts and photosensitizers;
(1c). the first phase solution with the quantity of flow Q₁ and the second phase solution with the quantity of flow Q₂ are mixed in the combined phase. Under turbulent shear, the two phase solutions mix rapidly to form a mixed solvent of the first phase and the second phase, and produce a stably dispersed composite nano formulation of photosensitizer and anti-tumor drug with a certain particle size and distribution coefficient; or,
method II: (2a). one or more of the anti-tumor drugs or their pharmaceutically acceptable salts are dissolved in first phase solvent to form first phase solution, and the first phase solvent is good solvent for anti-tumor drugs or their pharmaceutically acceptable salts;
(2b). one or more of the photosensitizers are dissolved in second phase solvent to form second phase solution, and the second phase solvent is anti-solvent for anti-tumor drugs or their pharmaceutically acceptable salts;
(2c). the first phase solution with quantity of flow Q₁ and the second phase solution with quantity of flow Q₂ are mixed in the combined phase. Under turbulent shear, the two phase solutions mix rapidly to form a mixed solvent of the first phase and the second phase, and produce a stably dispersed composite nano formulation of photosensitizer and anti-tumor drug with a certain particle size and distribution coefficient.

8. The method of claim 6, wherein the quantity of flow Q₁ of the first phase solution through the first pipeline is selected from 1-1000 ml/min; the temperature T₁ of the first phase solution is selected from 0-90° C.; the quantity of flow Q₂ of the second phase solution through the second pipeline is selected from 10-10000 ml/min; the temperature T₂ of the second phase solution is selected from 0-90° C.

9. The method of claim 6, wherein the production method fulfills one or more of the following:
(1) the temperature of the first phase solution is 0-90° C.;
(2) the temperature of the second phase solution is 0-90° C.;
(3) the Reynolds number Re of fluid in the combined phase is 800 to 7700;
(4) the flow velocity ratio FVR between the first phase solution and the combined phase is 5 to 26;
(5) when the Re of the combined phase is less than 3000, the FVR of the production system is 17 to 26, and/or the production system further includes a static mixer;
(6) when the FVR of the production system is less than 17, the Re of the combined phase is 3000 to 7700, and/or the production system further includes a static mixer.

10. The method of claim 6, wherein the production method fulfills one or more of the following:
(1) the temperature of the first phase solution is 25° C.;
(2) the temperature of the second phase solution is 25° C.;
(3) the Reynolds number Re of fluid in the combined phase is 3000 to 7700;
(4) the flow velocity ratio FVR between the first phase solution and the combined phase is 17 to 26;
(5) when the Re of the combined phase is less than 3868, the FVR of the production system is 20.8 to 26, and/or the production system further includes a static mixer;
(6) when the FVR of the production system is less than 20.8, the Re of the combined phase is 3868 to 7659, and/or the production system further includes a static mixer.

11. The method of claim 6, wherein the production method fulfills one or more of the following:
(1) the photosensitizer is selected from one or more of cyanine type molecules, porphyrin type molecules, porphyrin precursors, phthalocyanine type molecules and chlorin type molecules;
(2) the anti-tumor drug contains one or more of aromatic rings or aromatic heterocycles in its structure. The aromatic rings or aromatic heterocycles have conjugated planar ring systems covered by delocalized π electrons. The anti-tumor drugs are selected from one or more of camptothecin type compounds, paclitaxel type compounds, anthracycline type compounds, targeted drugs or other anti-tumor drugs;
(3) the molar ratio of the anti-tumor drug or its pharmaceutically acceptable salt to the photosensitizer is (1-15):1;
(4) the solvent used in the first phase solution and the second phase solution is water, an aqueous buffer solution with a certain pH value, or an organic solvent miscible with water. Further, the organic solvent is one or more of methanol, ethanol, propanol, isopropanol, butanol, isobutanol, tert-butanol, DMF, DMAc, N-methylpyrrolidone, DMSO, butyl sulfone, tetramethylene sulfone, THF, 2-methyltetrahydrofuran, acetonitrile, acetone, ethylene glycol, ethylene glycol methyl ether, ethylene glycol ethyl ether, HMPA, dioxane, formic acid, acetic acid, hydroxypropionic acid, ethylamine, ethylenediamine, glycerol or pyridine;
(5) the molar concentration of the anti-tumor drug or its pharmaceutically acceptable salt in the first phase solution is 0.01-0.3M;
(6) the molar concentration of the photosensitizer in the first phase solution or the second phase solution is 0.01-0.3M.

12. The method of claim 11, wherein the production method fulfills one or more of the following:
(1) the cyanine type molecules are selected from one or more of IR780, IR820, indocyanine green and indocyanine green analogs;
(2) the porphyrin type molecules are selected from hematoporphyrin monomethyl ether;
(3) the porphyrin precursors are selected from 5-aminolevulinic acid and/or 5-aminolevulinic acid esters;
(4) the phthalocyanine type molecules are selected from one or more of copper phthalocyanine, cobalt phthalocyanine, aluminum phthalocyanine, nickel phthalocyanine, calcium phthalocyanine, sodium phthalocyanine, magnesium phthalocyanine, zinc phthalocyanine, indium phthalocyanine, oxytitanium phthalocyanine, manganese phthalocyanine or phthalocyanine derivatives;
(5) the chlorin type molecules are selected from one or more of chlorins, talaporfin, verteporfin, temoporfin, rostaporfin, porfimer sodium, hemoporfin and HPPH.
(6) the camptothecin type molecules are selected from one or more of camptothecin, 9-aminocamptothecin, 9-nitrocamptothecin, lurtotecan, gimatecan, belotecan, 10-hydroxycamptothecin, SN-38, exatecan, irinotecan, topotecan and deruxtecan;
(7) the paclitaxel type compounds are selected from one or more of paclitaxel, docetaxel, cabazitaxel, 7-epipaclitaxel, 2′-acetylpaclitaxel, 10-deacetylpaclitaxel, 7-epi-10-deacetyltaxol, 7-xylosyltaxol, 10-deacetyl-7-glutarylpaclitaxel, 7-N,N-dimethylglycylpaclitaxel, 7-L-alanylacetaxel and larotaxel;
(8) the anthracycline type compounds are selected from one or more of doxorubicin, epirubicin, daunorubicin, pirarubicin and aclacinomycin;
(9) the targeted drugs are selected from one or more of gefitinib, erlotinib, lapatinib, afatinib, dacomitinib, vandetanib, neratinib, osimertinib, imatinib, sorafenib, sunitinib, lapatinib, dasatinib, olaparib, niraparib, rucaparib, fluzoparib, pamiparib, veliparib, talazoparib and apatinib;
(10) the other anti-tumor drugs are selected from one or more of etoposide, teniposide, vinblastine, vincristine, vinorelbine, vindesine, maytansine, curcumin, harringtonine, homoharringtonine, gemcitabine, capecitabine, fludarabine, cladribine, pemetrexed, bortezomib, carfilzomib, ixazomib, carmustine, fluorouracil, cytarabine, cyclosporine A, eribulin and trabectedin;
(11) the solvent in the first phase solution is selected from one or more of methanol, ethanol, propanol, isopropanol, butanol, isobutanol, tert-butanol, DMF, DMAc, N-methylpyrrolidone, DMSO, butyl sulfone, tetramethylene sulfone, THF, 2-methyltetrahydrofuran, acetonitrile, acetone, ethylene glycol, ethylene glycol methyl ether, ethylene glycol ethyl ether, HMPA, dioxane, formic acid, acetic acid, hydroxypropionic acid, ethylamine, ethylenediamine, glycerol and pyridine;
(12) the solvent in the second phase solution is water or a buffer with a pH of 2˜10;
(13) the molar concentration of the anti-tumor drug or its pharmaceutically acceptable salt in the first phase solution is 0.05-0.1M;
(14) the molar concentration of the photosensitizer in the first phase solution or the second phase solution is 0.05-0.1M.

13. The method of claim 6, wherein the production method fulfills one or more of the following:
(1) the photosensitizer is indocyanine green or chlorin e6;
(2) the anti-tumor drug is camptothecin, 10-hydroxycamptothecin, exatecan, Dxd, paclitaxel, sorafenib or curcumin.

14. The method of claim 6, wherein in the photosensitizer/anti-tumor drug composite nanoparticles, the combination of the photosensitizer and the anti-tumor drug is a combination of indocyanine green and camptothecin, indocyanine green and 10-hydroxycamptothecin, indocyanine green and 7-ethylcamptothecin, indocyanine green and 7-ethyl-10-hydroxycamptothecin, indocyanine green and exatecan, indocyanine green and Dxd, indocyanine green and paclitaxel, indocyanine green and docetaxel, indocyanine green and cabazitaxel, indocyanine green and sorafenib, indocyanine green and curcumin, chlorin e6 and 7-ethyl-10-hydroxycamptothecin;
the preferable combination of the photosensitizer and the anti-tumor drug is a combination of indocyanine green and camptothecin, indocyanine green and 10-hydroxycamptothecin, indocyanine green and exatecan, indocyanine green and Dxd, indocyanine green and paclitaxel, indocyanine green and sorafenib, indocyanine green and curcumin, indocyanine green and 7-ethyl-10-hydroxycamptothecin, chlorin e6 and 7-ethyl-10-hydroxycamptothecin.

15. The method of claim 6, wherein the production method fulfills one or more of the following:
(1) when the anti-tumor drug is 7-ethyl-10-hydroxycamptothecin and the photosensitizer is indocyanine green, the molar ratio of the anti-tumor drug or its pharmaceutically acceptable salt to the photosensitizer is (2-15):1;
(2) when the anti-tumor drug is camptothecin and the photosensitizer is indocyanine green, the molar ratio of the anti-tumor drug or its pharmaceutically acceptable salt to the photosensitizer is (1-10):1;
(3) when the anti-tumor drug is 10-hydroxycamptothecin and the photosensitizer is indocyanine green, the molar ratio of the anti-tumor drug or its pharmaceutically acceptable salt to the photosensitizer is (1-10):1;
(4) when the anti-tumor drug is exatecan and the photosensitizer is indocyanine green, the molar ratio of the anti-tumor drug or its pharmaceutically acceptable salt to the photosensitizer is (2-15):1;
(5) when the anti-tumor drug is Dxd and the photosensitizer is indocyanine green, the molar ratio of the anti-tumor drug or its pharmaceutically acceptable salt to the photosensitizer is (5-10):1;
(6) when the anti-tumor drug is sorafenib and the photosensitizer is indocyanine green, the molar ratio of the anti-tumor drug or its pharmaceutically acceptable salt to the photosensitizer is (6-8):1;
(7) when the anti-tumor drug is paclitaxel and the photosensitizer is indocyanine green, the molar ratio of the anti-tumor drug or its pharmaceutically acceptable salt to the photosensitizer is (1-10):1;
(8) when the anti-tumor drug is curcumin and the photosensitizer is indocyanine green, the molar ratio of the anti-tumor drug or its pharmaceutically acceptable salt to the photosensitizer is (5-8):1;
(9) when the anti-tumor drug is 7-ethyl-10-hydroxycamptothecin and the photosensitizer is chlorin e6, the molar ratio of the anti-tumor drug or its pharmaceutically acceptable salt to the photosensitizer is 2:1.

16. The method of claim 6, wherein the production method also includes the following post-reaction processing steps: adding water to the prepared mixed solution and ultrafiltration; or further concentration and ultrafiltration.
wherein, the ultrafiltration uses 2-100 kDa ultrafiltration membrane.

17. The method of claim 6, wherein the production method fulfills one or more of the following:
(1) the particle size of the nanoparticles is less than 1000 nm;
(2) the polydispersity index of the nanoparticle is less than 0.4.

18. A photosensitizer/anti-tumor drug composite nanoparticle, which is prepared by the method of claim 6; the photosensitizer/anti-tumor drug composite nanoparticle is SN-38/ICG composite nanoparticles, wherein the SN-38 encapsulation efficiency is greater than 80%.

19. The system of claim 4, wherein the static mixer is selected from one or more of SV type static mixer, SX type static mixer, SL type static mixer, SH type static mixer and SK type static mixer.

20. The method of claim 6, wherein the production method fulfills one or more of the following:
(1) the Reynolds number Re of fluid in the combined phase is 3868 to 7659;
(2) the flow velocity ratio FVR between the first phase solution and the combined phase is 20.8 to 26;
(3) when the anti-tumor drug is 7-ethyl-10-hydroxycamptothecin and the photosensitizer is indocyanine green, the molar ratio of the anti-tumor drug or its pharmaceutically acceptable salt to the photosensitizer is 2:1, 5:1, 10:1 or 15:1;
(4) when the anti-tumor drug is camptothecin and the photosensitizer is indocyanine green, the molar ratio of the anti-tumor drug or its pharmaceutically acceptable salt to the photosensitizer is 1:1, 2:1, 5:1 or 10:1;
(5) when the anti-tumor drug is 10-hydroxycamptothecin and the photosensitizer is indocyanine green, the molar ratio of the anti-tumor drug or its pharmaceutically acceptable salt to the photosensitizer is 1:1, 2:1, 5:1 or 10:1;
(6) when the anti-tumor drug is exatecan and the photosensitizer is indocyanine green, the molar ratio of the anti-tumor drug or its pharmaceutically acceptable salt to the photosensitizer is (2-10): 1;
(7) when the anti-tumor drug is paclitaxel and the photosensitizer is indocyanine green, the molar ratio of the anti-tumor drug or its pharmaceutically acceptable salt to the photosensitizer is 1:1, 2:1, 5:1 or 10:1;
(8) when the anti-tumor drug is curcumin and the photosensitizer is indocyanine green, the molar ratio of the anti-tumor drug or its pharmaceutically acceptable salt to the photosensitizer is 5:1, 6:1, 7:1 or 8:1;
(9) the ultrafiltration uses 30 kDa ultrafiltration membrane;
(10) the particle size of the nanoparticles is less than 500 nm.
